(19) United States
(12) Patent Application Publication
(10) Pub. No.: US 2019/0298664 A1
(43) Pub. Date: Oct. 3, 2019

(54) METHOD AND KIT FOR MYOCARDIAL REPERFUSION, AND METHOD FOR ATTENUATING OR REDUCING MYOCARDIAL REPERFUSION INJURY

(71) Applicants: Academia Sinica, Taipei (TW); Buddhist Tzu Chi Medical Foundation, Hualien (TW)

(72) Inventors: Tzu-Ching Meng, Taipei (TW); Chiu-Fen Yang, Taipei (TW); Michael Yu-Chih Chen, Hualien (TW)

(73) Assignees: Academia Sinica, Taipei (TW); Buddhist Tzu Chi Medical Foundation, Hualien (TW)

(21) Appl. No.: 16/368,654

(22) Filed: Mar. 28, 2019

Related U.S. Application Data
(60) Provisional application No. 62/649,201, filed on Mar. 28, 2018.

(51) Int. Cl. A61K 31/10; A61K 33/242; A61K 31/727; A61K 45/06; A61K 9/00; A61P 9/10

(52) U.S. Cl. CPC A61K 31/10; A61K 33/242; A61P 9/10; A61K 45/06; A61K 9/0019; A61K 31/727

(57) ABSTRACT

This disclosure provides method and kit for myocardial reperfusion and method for attenuating or reducing myocardial reperfusion injury. By administering protein tyrosine phosphatases inhibitor or a protein tyrosine kinases activator, cardiac injury caused by ischemia/reperfusion will be attenuated or reduced.

### CROSS-REFERENCE TO RELATED APPLICATION

This application claims the benefit of U.S. Provisional Application No. 62/649,201, filed Mar. 28, 2018.

### FIELD OF INVENTION

This present disclosure relates, in general, to the prevention and/or treatment of ischemia/reperfusion injury; particularly, to the use of a protein tyrosine phosphatases (PTPs) inhibitor, a protein tyrosine kinases (PTKs) activator, or a combination thereof, in the prevention and/or treatment of ischemia/reperfusion injury.

### BACKGROUND OF THE INVENTION

Acute myocardial infarction and its consequences are still significant health problems regardless of the continuing development of new drugs and improvement of medical care in the recent years. To date, reperfusion remains the only established clinical strategy to salvage the injured hearts and decline patients' mortality and morbidity. However, reperfusion per se also cause additional damage to the ischemic myocardium [1].

Myocardial reperfusion injury, first reported by Jennings et al. in 1960 [2], was characterized by cell swelling, contracture of myofibrils, and disruption of sarcolemma in the myocardium already under the ischemic stress. Over the last few decades, there has been a significant expansion of the scientific findings that clearly define the myocardial damage induced by ischemia/reperfusion (I/R). Four types of detrimental challenges, including myocardial stunning, coronary no-reflow phenomenon, reperfusion arrhythmia and lethal reperfusion injury [1], collectively lead to threatened death of the cardiomyocytes after an onset of reperfusion. The episode of these insults could significantly diminish the benefit of reperfusion therapy. In order to conquer the I/R injury, a tremendous effort has been devoted to investigate the underlying mechanism of such pathological process and to develop experimental therapeutics.

However, the translation of these findings into a practical application for patient care has been largely disappointing. New interventions that prevent myocardial injury caused by I/R are still the unmet clinical need. Obviously, an innovative treatment of AMI patients with a new strategy that may reduce the reperfusion injury must be developed in order to fulfill the unmet clinical need.

### SUMMARY OF THE INVENTION

In view of the urgent need of the art, provided herein are pharmacological strategies that are safe and effective in the treatment of the ischemia/reperfusion injury.

In the present application, we develop a method for attenuating or reducing ischemia/reperfusion-related damages, in which protein tyrosine phosphatases (PTPs) is inhibited or protein tyrosine kinases (PTKs) is activated. Preferably, the protein tyrosine phosphatases comprises PTP-PEST. In one embodiment, the beneficial effect of auranofin, was demonstrated in managing cardiac I/R injury.

In one aspect, provided herein is a method for attenuating or reducing myocardial reperfusion injury in a subject with ischemia myocardium, comprising: administering to the subject an effective amount of a protein tyrosine phosphatases (PTPs) inhibitor or a protein tyrosine kinases (PTKs) activator. Preferably, the subject may be human or animal.

In another aspect, provided herein is a method for myocardial reperfusion, comprising administering to a subject in need an effective amount of a protein tyrosine phosphatases (PTPs) inhibitor or a protein tyrosine kinases (PTKs) activator, and performing a reperfusion therapy on the subject.

Preferably, the subject may have an elevated myocardial PTPs activity during ischemia and reperfusion. More preferably, the subject may have a decreased myocardial protein tyrosine phosphorylation.

Preferably, the PTPs inhibitor may be a PTP-PEST inhibitor and may comprise auranofin, phenyl vinyl sulfone (PVS), or orthovanadate. More preferably, an IC₅₀ of the auranofin to the PTP-PEST may be 38.7 μM.

Preferably, a phosphorylation on Paxillin, p130cas, and ErbB-2 of the subject may be restored after the administration. More preferably, the restoration of the phosphorylation may be at Y118 of Paxillin, Y410 of p130cas, and Y1248 of ErbB-2.

Preferably, the myocardial reperfusion injury may comprise cellular swelling and necrosis, apoptosis, edema, hemorrhage, the no-reflow phenomenon, tissue damage by free oxygen radicals, contracture of myofibrils or disruption of sarcolemma in a myocardium under ischemic stress.

Preferably, the PTPs inhibitor may be administered before or during ischemia. Alternatively, the PTPs inhibitor may be administered before reperfusion.

Preferably, the administration may be by at least one mode selected from the group consisting of parenteral, subcutaneous, intra-muscular, intra-venous, intra-articular, intra-bronchial, intra-abdominal, intra-capsular, intra-cartilaginous, intra-cavitary, intra-celial, intra-cerebellar, intra-cerebroventricular, intra-colic, intra-cervical, intra-gastric, intra-hepatic, intra-myocardial, intra-osteal, intra-pelvic, intra-pericardiac, intra-peritoneal, intra-pleural, intra-prostatic, intra-pulmonary, intra-rectal, intra-renal, intra-retinal, intra-spinal, intra-synovial, intra-thoracic, intra-uterine, intra-vesical, bolus, vaginal, rectal, buccal, sublingual, intra-nasal, transdermal, and intra-coronary.

Preferably, the effective amount may be in a range of 0.01-80, 0.05-40 or 0.1-10 mg/kg.

Preferably, a serum troponin I level and an infarct size of the subject may be reduced after the administration.

Preferably, an inflammatory cells infiltration, fibrosis and myocardial thinning of the subject may be reduced after the administration.

Preferably, the PTPs inhibitor or the PTKs activator may further comprise a pharmaceutically acceptable carrier such as solvent, emulsifier, suspending agent, decomposer, binding agent, excipient, stabilizing agent, chelating agent, diluents, gelling agent, preservative, lubricant, or a combination thereof.

Preferably, the PTPs inhibitor or the PTKs activator may be in a form selected from the group consisting of a solution, a suspension, a gel and an ointment.

In one aspect, provided herein is a kit for myocardial reperfusion, comprising: a protein tyrosine phosphatases (PTPs) inhibitor or a protein tyrosine kinases (PTKs) activator, and a means for myocardial reperfusion.

Preferably, the PTPs inhibitor may comprise PTP-PEST inhibitor. More preferably, the PTP-PEST inhibitor may comprise auranofin, or phenyl vinyl sulfone (PVS), or orthovanadate.

Preferably, the means for myocardial reperfusion may be thrombolytic agents or fibrinolytic agents for thrombolysis. More preferably, the thrombolytic agents or the fibrinolytic agents may comprise streptokinase, urokinase, alteplase, reteplase, tenecteplase, or recombinant tissue plasminogen activator (rtPA). In addition, the kit may further comprise an anticoagulant including heparin or low molecular weight heparin.

Preferably, the means for myocardial reperfusion may be selected from the group consisting of a stent, a balloon, aspiration thrombectomy, rotational atherectomy, laser angioplasty, cutting balloon angioplasty, and embolic protection device and any combinations thereof, for a percutaneous coronary intervention (PCI) and a coronary angioplasty.

Preferably, the means for myocardial reperfusion is surgical equipments for bypass surgeries that graft arteries around blockages.

### BRIEF DESCRIPTION OF THE DRAWINGS

The patent or application file contains at least one color drawing. Copies of this patent or patent application publication with color drawing will be provided by the USPTO upon request and payment of the necessary fee.

FIGS. 1A-1H illustrate that inhibition of myocardial PTP activity prevents the mouse heart from I/R injury. (A) The PTP activity (orthovanadate-sensitive fraction) in the mouse heart extracts was examined using pNPP as the substrate (n=4-5). (B) Aliquots of the mouse heart extracts were probed with anti-pTyr antibody. (C) The experimental design illustrates how the treatment with vehicle control or PVS was performed in the mice subjected to I/R. (D-E) Aliquots of the mouse heart extracts were probed with anti-pTyr antibody. Shown are a representative blot (D) and the quantitative results (E; n=5). (F) Representative cardiac Evans blue/TTC staining (infarct area, white; area at risk, white and red; non-occluded area, blue) with the quantification of infarct size (G; n=6-8), and (H) serum troponin I from I/R mice treated with vehicle control and PVS (n=6-8), are depicted. I: ischemia 1 h, I/R: ischemia 1 h followed by reperfusion 4 h. (A), (E), (G) and (H), *P<0.05; **P<0.01; ***P<0.001.

FIGS. 2A-2F illustrate that RNAi ablation of PTP-PEST protects the neonatal cardiomyocytes against the H/R-induced cell damage. (A) The RNAseq analysis revealed the top 15 PTPs expressed in the mouse myocardium (GSM929707). The expression level was presented by the RPKM value. (B) The experimental design illustrates how the effect of PTP RNAi on hypoxia/reoxygenation (H/R)-induced injury in neonatal cardiomyocytes was performed. (C) Aliquots of total lysates from RNAi-ablated cardiomyocytes were probed with indicated antibodies. (D) Representative images show the effect of RNAi ablation on H/R-induced morphology change in neonatal cardiomyocytes (green: troponin T; blue: DAPI). (E-F) The H/R-induced cell injury was monitored by immunofluorescence intensity (E) and LDH released to the culture medium (F) in RNAi-ablated cells under H/R (n=5). *P<0.05, compared to control siRNA.

FIGS. 3A-3H illustrate that the cleaved and activated form of myocardial PTP-PEST appears in the mouse heart under I/R. (A) The schematic illustration shows the proposed mechanism for PTP-PEST activation. (B) The epitope of antibodies used to probe PTP-PEST. The strain D4W7W can detect the full-length PTP-PEST and the clone 2530 can recognize the N-terminal fragment of PTP-PEST upon cleavage by Caspase-3 at DSPD552. (C-D) shows the representative immunoblotting from the aliquots of mouse heart extracts probed with the indicated antibodies (C) and the results of quantification (D). The heart extracts were probed with the antibody recognizing the specific pTyr residue of Paxillin or p130cas (E) or ErbB-2 (G). The quantitative results of pTyr level were normalized to the corresponding protein expression (F and H; n=5). S: sham operation control; I/R: ischemia 1 h followed by reperfusion 4 h. **P<0.01 vs. sham control.

FIGS. 4A-4G illustrate that auranofin destabilizes the catalytic domain of PTP-PEST allosterically, thus inhibiting its enzymatic activity. (A) In vitro assay determined the IC50 of Auranofin to the purified PTP-PEST catalytic domain using synthetic phospho-Paxillin peptide as the substrate. (B) The experimental workflow illustrates how the effect of Auranofin on dynamic conformation of PTP-PEST catalytic domain was performed by HDX-MS. (C) Structure mapping of the relative deuterium fractional uptake levels on PTP-PEST in the absence (−, the apo form) and presence (+) of Auranofin treatment. The representations are color ramped from blue to red, corresponding to 0 to 60% fractional deuterium uptake as indicated by the scale bar shown on the top. (D) Shown is the subtractive difference of fractional deuterium uptake between apo and Auranofin-treated PTP-PEST. The color ramped from red to blue represents the degree of structure flexibility as indicated by the scale bar marked on the top. (C and D) The active-site cysteine (C231) is marked in yellow. (E) The analysis of DSF (left) or DSC (right) shows the Tm value of apo and Auranofin-treated PTP-PEST. (F and G) The H9c2 cells were treated by Auranofin with indicated concentrations. (F) The PTP activity (orthovanadatesensitive fraction) was assessed by the assay using pNPP as a substrate (n=5). **P<0.01 vs. 0 μM. (G) Aliquots of H9c2 total lysates were probed with the indicated antibodies (n=8). **P<0.01 vs. vehicle control.

FIGS. 5A-5E illustrate that auranofin inhibits myocardial PTP activity, leading to increased pTyr levels of PTP-PEST substrates in the mouse heart under FR. (A) The experimental design illustrates how the treatment with vehicle control or Auranofin was performed in the mice subjected to I/R. (B-E) Results were obtained from the mice with the indicated treatments for four hours post-I/R. (B) The PTP activity (orthovanadate-sensitive fraction) in the mouse heart extracts was examined by the assay using pNPP as a substrate (n=6). (C) Aliquots of the mouse heart extracts were probed with anti-pTyr antibody. Red arrowheads indicate the signals with a prominent change. (D and E) Shown are the representative immunoblotting and quantitative results of the heart extracts probed with the antibody against the specific pTyr residue of Paxillin or p130cas (D; n=5) and ErbB-2 (E; n=5-6). S: sham operation control, I/R: I/R-mice treated with the vehicle control, I/R+A: I/R-mice treated with the Auranofin. *P<0.05, **P<0.01.

FIGS. 6A-6H illustrate that the treatment of Auranofin attenuates the myocardial injury and improves the cardiac function in the mouse heart one day post-I/R. The experimental design was illustrated in FIG. 5A. (A-C) Shown are the representative cardiac Evans blue/TTC staining (A, infarct area, white; area at risk, white and red; non-occluded area, blue) with the quantification of infarct size (B; n=8), and serum troponin I (C; n=9) from the mice with the indicated treatments for 4 hours post-I/R. (D) Representative echocardiographic images and (E) the quantification of ejection fraction obtained from the mice with the indicated treatments for 24 hours post-I/R (n=8). (F-H) Shown are the representative cardiac Evans blue/TTC staining (F, infarct area, white; area at risk, white and red; non-occluded area, blue) with the quantification of infarct size (G; n=8), and serum troponin I (H; n=9) from the mice with the indicated treatments for 24 hours post-I/R. S: sham operation control, I/R: I/R-mice treated with the vehicle control, I/R+A: I/R-mice treated with Auranofin. (B), (C), (E), (G) and (H), **P<0.01.

FIGS. 7A-7F illustrate that the treatment of Auranofin maintains the cardiac performance in the mouse heart one week post-I/R. The experimental design was illustrated in FIG. 5A. (A) The representative echocardiographic images, (B) the quantification of ejection fraction, (C) the representative histopathologic H&E statin and (D) the quantification of infarct size were obtained from the mice with the indicated treatments for one week post-I/R (n=8). (E) The representative enlarged H&E stain obtained from mice one week post-I/R. The areas highlighted in yellow boxes (left panel) are shown by the enlarged view (right panel). (F) The representative Masson Trichrome stain obtained from mice one week post-I/R. The black arrows indicate the location of inflammation or fibrosis in the myocardium. I/R: I/R-mice treated with the vehicle control, I/R+A: FR-mice treated with Auranofin. (B) and (D) **P<0.01.

### DETAILED DESCRIPTION

The foregoing and other aspects of the present disclosure will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process, or method.

The transitional phrase “consisting of” excludes any elements, steps, or ingredients not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

Where applicants have defined an invention or a portion thereof with an open-ended term such as “comprising,” it should be readily understood that (unless otherwise stated) the description should be interpreted to also describe such an invention using the term “consisting of.”

As used herein, the term “about” is used to indicate that a value includes for example, the inherent variation of error for a measuring device, the method being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive, although the disclosure supports a definition that refers to only alternatives and “and/or.”

The use of the terms “treating,” or “treatment” is referred to herein as administration of a therapeutic composition to a subject with the purpose to cure, alleviate, relieve, remedy, prevent, or ameliorate a disorder, symptoms of the disorder, a disease state secondary to the disorder, or predisposition toward the disorder. The terms “inhibiting,” “reducing,” or “prevention,” or any variation of these terms, when used in the claims and/or the specification includes any measurable decrease or complete inhibition to achieve a desired result.

“Subject” as used herein refers to human or animals, including, for example, a mammalian subject diagnosed with or suspected of having or developing cardiovascular disease, particularly, myocardial infarction. Exemplary subject may be humans, apes, dogs, pigs, cattle, cats, horses, goats, sheep, rodents and other mammalians with the diseases that can benefit from the treatment.

“Administering” or “Administration” is referred to herein as providing a treatment kit of the present application to a subject. By way of example and not limitation, administration may be performed via parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal. For example, injection may be performed by intravenous (i.v.) injection, sub-cutaneous (s.c.) injection, intradermal (i. d.) injection, intraperitoneal (i.p.) injection, or intramuscular (i. m.) injection. One or more such routes may be employed. Parenteral administration can be, for example, by bolus injection or by gradual perfusion over time. Alternatively, or concurrently, administration may be by the oral route.

In certain embodiments, it is desired to limit, reduce, or ameliorate infarct size and/or reverse or reduce reperfusion injury. The routes of administration will vary, naturally, with the location and nature of the lesion or site to be targeted, and include, e.g., regional, parenteral, intravenous, intramuscular, and/or systemic administration and formulation. Direct injection or injection into the vasculature or the vessels to and from and within an organ or tissue is specifically contemplated for target areas. Local, regional, or systemic administration also may be appropriate.

As used herein, the term “inhibitor” or “activator” means a molecular entity of natural, semi-synthetic or synthetic origin that either activates or blocks, stops, inhibits, and/or suppresses the protein tyrosine phosphatases (PTPs) and the protein tyrosine kinases (PTKs). For instance, the activator will activate the pathway while the inhibitor will block, stop, inhibit, and/or suppress a pathway.

Unless otherwise defined, all technical and scientific terms used herein have the same meaning as commonly understood by one of ordinary skill in the art to which this invention belongs. All publications, patent applications, patents and other references cited herein are incorporated by reference in their entireties for the teachings relevant to the sentence and/or paragraph in which the reference is presented.

Ischemia is a deficiency of blood or blood flow in a part typically due to functional constriction or actual obstruction of a blood vessel. Such a deficiency result in an infarct, an area of cell death in a tissue due to local ischemia resulting from obstruction of circulation to the area, most commonly by a thrombus, embolus, or ruptured or obstructing atherosclerotic plaque. When the constriction or obstruction is removed and blood flow restored reperfusion occurs. Although blood flow is restored, the reperfusion can also result in adverse effects including cellular swelling and necrosis, apoptosis, edema, hemorrhage, the no-reflow phenomenon, and tissue damage by free oxygen radicals.

Reperfusion injury to the heart is accompanied by the upregulation and post-translational modification of a number of proteins normally involved in regulating cell cycle progression. Disclosed are methods for treating, ameliorating, reducing, and/or limiting reperfusion injury including, but not limited to reduction or limitation of infarct size. In certain aspects, the methods are equally appropriate for use in reducing I/R injury including, but not limited to ischemic strokes (including strokes resulting from cerebral thrombosis, cerebral embolism, and atrial fibrillation), hemorrhagic strokes (including strokes resulting from aneurysm and arteriovenous malformation), and transient ischemic attack), reducing infarct size following pulmonary infarction, reducing renal ischemia injury, reducing ischemic/reperfusion injury occurring during cardiac surgery where a heart lung machine is used such as coronary artery bypassing, and reducing reperfusion injury occurring during the preservation of organs for transplant.

Disclosed are a method for attenuating or reducing myocardial reperfusion injury in a subject with ischemia myocardium, comprising: administering to the subject an effective amount of a protein tyrosine phosphatases (PTPs) inhibitor or a protein tyrosine kinases (PTKs) activator. One manifestation of reducing reperfusion injury is reducing or limiting or ameliorating infarct size. Therefore, disclosed herein are methods of reducing infarct size following a reperfusion event in a subject comprising administering to the subject the inventive compositions that inhibit, reduce, limit, or ameliorate an infarct.

PTPs inhibitors may comprise but not limit to quinolyl, cyclic alabenzimidazole, pyrazine, (ethynediyl)bis-benzene, pyridopyrimidine, triazolopyridine, cyclo propylphenyl phenyloxamides, oxindole or azoloarin derivatives, which discussed in detail in Sobhia et al. (Expert Opinion on Therapeutic Patents, 22(2), 125-153, 2012), which is hereby incorporated into this specification by reference. In one embodiments, PTPs inhibitor may be a PTP-PEST inhibitor. In another embodiment, PTPs inhibitor may comprise auranofin, phenyl vinyl sulfone (PVS), or orthovanadate. PTKs activator may comprise but not limit to ligands such as growth factors or hormone, for example, platelet-derived growth factor (PDGF), epidermal growth factor (EGF), or insulin.

By “treatment” and “treating” is meant the medical management of a subject with the intent to cure, ameliorate, stabilize, or prevent a disease, condition, or disorder. This term includes active treatment, i.e, treatment directed specifically toward the improvement of a disease, condition, or disorder. Treatment and treating also include causal treatment, i.e., treatment directed toward removal of the cause of the associated disease, condition, or disorder. In addition, this term includes palliative treatment, i.e., treatment designed for the relief of symptoms rather than the curing of the disease, condition, or disorder; preventative treatment, i.e., treatment directed to minimizing or partially or completely inhibiting the development of the associated disease, condition, or disorder; and supportive treatment, i.e., treatment employed to supplement another specific therapy directed toward the improvement of the associated disease, condition, or disorder. It is understood that treatment, while intended to cure, ameliorate, stabilize, or prevent a disease, condition, or disorder, need not actually result in the cure, ameliorization, stabilization, or prevention. It is understood and herein contemplated that “treatment” does not necessarily refer to a cure of the disease or condition nor a complete prevention of infarct, but can involve, for example, an improvement in the outlook of a reperfusion injury. The effects of treatment can be measured or assessed as described herein and as known in the art as is suitable for the disease, condition, or disorder involved (e.g., MI, etc.). Such measurements and assessments can be made in qualitative and/or quantitative terms. Thus, for example, characteristics or features of a disease, condition, or disorder and/or symptoms of a disease, condition, or disorder can be reduced to any effect or to any amount.

Treatment regimens may vary as well and often depend on target site, subject condition, and health and age of the SUBJECT. Certain conditions will require more aggressive treatment. The clinician will be best suited to make such decisions based on the known efficacy and toxicity (if any) of the therapeutic formulations or methods.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. A unit dose may conveniently be described in terms of ng, or mg of component. Alternatively, the amount specified may be the amount administered per subject weight (typically kg) or as the average daily, average weekly, or average monthly dose.

Also, for example, treating reperfusion injury can comprise any method or the administration of any combination of a PTPs inhibitor and a PTKs activator that affects tissue damage resulting from reperfusion or ameliorates the degree of or potential for tissue injury associated with an ischemia/reperfusion event.

“Reducing,” “reduce,” or “reduction” in the context of a disease or condition herein refers to a decrease in the cause, symptoms, or effects of a disease or condition. Therefore, in the disclosed methods, “reducing” can refer to a 10%, 20%, 30%, 40%, 50%, 60%, 70%, 80%, 90%, or 100% decrease, or any value or range there between, in the amount of injury due to reperfusion including but not limited to infarct size.

In certain embodiments the composition comprising a PTPs inhibitor and/or a PTKs activator is administered before the ischemia and/or reperfusion event. Thus, it is contemplated that subject at risk for or having a history of ischemia/reperfusion events can decrease the risk of further necrosis in future events by administration of a PTPs inhibitor and/or a PTKs activator prophylactically, which also includes prior to, during, or after catheterization or other medical procedures. It is also understood that many ischemia/reperfusion events have early warning symptoms preceding the actual event which when recognized can allow the subject to seek immediate treatment. Even if there is ischemic/reperfusion injury caused by future ischemia/reperfusion events, it is contemplated that the prophylactic administration of a PTPs inhibitor and/or a PTKs activator will reduce infarct size. For example, disclosed herein are methods of reducing ischemia/reperfusion injury in a subject in need thereof (having, had, or at risk of having an ischemic/reperfusion event) comprising administering to the subject a PTPs inhibitor and/or a PTKs activator that, wherein the composition is administered at least 30 minutes before the ischemia/reperfusion event. Thus, disclosed herein are methods wherein the agent is administered 15, 30 minutes, 1, 2, 6, 12, 24 hour(s), 2, 3, 4, 5, 6 days, 1 or 2 weeks or any time points in between before the ischemia/reperfusion event.

In particular aspects, disclosed herein are methods for myocardial reperfusion, comprising administering to a subject in need an effective amount of a protein tyrosine phosphatases (PTPs) inhibitor or a protein tyrosine kinases (PTKs) activator, and performing a reperfusion therapy on the subject. PTPs inhibitors and/or PTKs activators can be administered before, during, and/or after reperfusion due to percutaneous transluminal coronary angioplasty, vascular grafts in surgical revascularization (before removal of the aortic cross-clamp in on-pump cardiac surgery), removal of the target vessel ligature during off-pump coronary artery bypass graft surgery, organ transplantation or other procedures of events that impede blood flow to myocardium or other organs or tissues.

The effective amount of the PTPs inhibitors and/or PTKs activators for attenuating or reducing myocardial reperfusion injury may be in a range of 0.01-80, 0.05-40 or 0.1-10 mg/kg body weight. Particularly, the effective amount is 0.01, 0.02, 0.05, 0.1, 0.2, 0.3, 0.4, 0.5, 0.6, 0.7, 0.8, 0.9, 1.0, 1.2, 1.5, 1.8, 2, 5, 8, 10, 15, 20, 25, 30, 35, 40, 45, 50, 55, 60, 65, 70, 75, or 80.

In particular aspects, disclosed herein is a kit for myocardial reperfusion, comprising: a protein tyrosine phosphatases (PTPs) inhibitor or a protein tyrosine kinases (PTKs) activator, and a means for myocardial reperfusion. The means for myocardial reperfusion may be chemical agents including thrombolytic agents or fibrinolytic agents for thrombolysis, or equipments including a stent, a balloon, an embolic protection device or those for performing aspiration thrombectomy, rotational atherectomy, laser angioplasty, cutting balloon angioplasty, bypass surgeries that graft arteries around blockages, percutaneous coronary intervention (PCI), coronary angioplasty and any combinations thereof.

In some embodiments, the method for the delivery of a PTPs inhibitor and/or a PTKs activator is via intraarterial or intravenous administration. Injection of a PTPs inhibitor and/or a PTKs activator may be delivered by syringe or catheter or any other method used for injection of a solution, as long as the PTPs inhibitor and/or a PTKs activator and any associated components can pass through the particular gauge of needle or device required for injection or intravascular delivery.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid- or oil-based. In particular embodiments of the invention, a composition comprising one or more PTPs inhibitor and/or PTKs activator is in a water-based formulation. In other embodiments, the formulation is lipid based.

For aqueous solutions, the solution should be suitably buffered if necessary. A liquid diluent is typically rendered isotonic with sufficient saline or glucose. These aqueous solutions are especially suitable for intravenous administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The PTPs inhibitor and/or the PTKs activator are administered in a manner compatible with the dosage formulation, and in such amount as will be therapeutically effective. The quantity to be administered depends on the subject to be treated, including, e.g., the length and severity of an ischemic event. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7 days or more.

It will be apparent to those skilled in the art that various modifications and variations can be made to the disclosed embodiments. It is intended that the specification and examples be considered as exemplary only, with a true scope of the disclosure being indicated by the following claims and their equivalents.

Material and Methods

Experimental Animals

Male C57BL/6JNarl mice (8-12 weeks) will be used in this study. The animals are kept in cages (maximum 5 mice per cage) in the animal center and fed with standard diet and tap water ad libitum on a 12-h/12-h light/dark cycle. All animal work is conducted in accordance with the guidelines of the Institutional Animal Care and Use Committee of Academia Sinica, Taipei, Taiwan.

Cardiac Ischemia and Reperfusion Procedure.

We anesthetized the mouse with 5% isoflurane mixed with room air until loss of sensation of pain and then maintained the mouse in anesthesia status with 2% isoflurane mixed with room air through the whole procedure. The mouse's body temperature was maintained at 37° C. by a heating pad. After the mouse was intubated, we provided artificial ventilation to the mouse with a small animal ventilator. Then the chest cavity was opened at the second intercostal space and homemade chest retractors were utilized to widen the incision. We pulled the pericardium apart carefully and exposed the left anterior descending artery (LAD). A 7-0 silk was put underneath the LAD at the position of the tip of the left auricle, a 6 mm length of PE-10 tube was put on the surface of the LAD, and then a double knot was made to occlude the blood flow of LAD. The success of ligation was confirmed by paling the anterior wall of the LV. For the reperfusion group, the LAD knot would be untied for the purpose of reperfusion after 1 hour of ischemia. For the sham operation group, all procedures were the same except for the knot that occluded the LAD flow. After the successful procedure, we left the silk in situ, closed the chest cavity and weaned the animal from the ventilator.

Reagents and Antibodies

Antibodies specific for PTP-PEST full-length (D4W7W), p-paxillin (Y118), p-p130cas (Y410), ErbB-2, p-ErbB2 (Y1248) and caspase-3 were purchased from Cell Signaling Technology. The antibody for PTP-PEST cleaved form was homemade rabbit polyclonal antibody, clone 2530. Antibodies against paxillin, PTP-alpha, and GAPDH were obtained from EMD Millipore. The antibody for p130cas was from BD. The chemicals were from Sigma and Invitrogen. The recombinant caspase-3 was made by BioVision. The kits for LDH measurement was purchased from TaKaRa.

Overall PTP Activity Measurement by pNPP Assay

For heart tissue: At the end of the treatment, the left ventricle of mouse heart is gently homogenized in ice-cold RIPA buffer with Ser/Thr phosphatase inhibitors inside. Then the samples are centrifuged at 16,100×g for 10 min at 4° C. to remove the debris. Tissue extract (1 mg) is incubated with pNPP (2 mM) in reaction buffer at 37° C. for 60 min on a shaker. At the end of incubation, the reaction is terminated by addition of the same volume of 0.1 N NaOH. The released p-nitrophenolate ion is measured by spectrophotometry reading the absorbance at 405 nm. For H9C2 cell: At the end of the treatment, the cells are collected in ice-cold RIPA buffer with Ser/Thr phosphatase inhibitors inside. Then the samples are centrifuged at 16,100×g for 10 min at 4° C. to remove the debris. The cell lysate is incubated with pNPP (2 M) in reaction buffer at 37° C. for 90 min on a shaker. The reaction is terminated by adding the same volume of 0.1 N NaOH. The released p-nitrophenolate ion is measured by spectrophotometry reading the absorbance at 405 nm.

Immunoblotting.

Hearts from mice received different treatment were gently homogenized in ice-cold RIPA buffer [250 mM Tris-HCl pH 7.4, 750 mM NaCl, detergent mix (5% NP-40, 2.5% Na-deoxycholate and 0.5% SDS), and 1× protease inhibitor cocktail (EDTA free)] containing phosphatase inhibitors (2 mM Na₃VO₄, 10 mM NaF, and 10 mM Na₂P₂O₇). The homogenates were centrifuged at 16,100×g at 4° C. for 10 minutes to remove the debris. Protein concentrations were determined by Bradford protein assay. Equal amounts of protein extract from those mice were separated by SDS-PAGE on an 8% or 10% polyacrylamide gel and then transferred to PVDF membrane. Membranes were incubated with indicated primary antibody at 4° C. overnight followed by incubated with secondary antibodies for 1 hour at room temperature. The protein abundance was detected by the signal intensity of each specific band.

In-Gel Phosphatase Assay

At the end of treatment, the mice hearts from sham and I/R group were harvested and gently homogenized in ice-cold lysis buffer [20 mM HEPES in pH 7.4, 1% NP-40, 2 mM EDTA, 5 mM DTT, and 1× protease inhibitor cocktail]. The homogenates were centrifuged at 16,100×g at 4° C. for 10 minutes and the supernatants were used as tissue extracts. Protein concentrations were determined by Bradford protein assay. Equal amounts of protein extracts (100 μg/lane) from those mice were separated by SDS-PAGE on a 10% polyacrylamide gel under 30 mA constant current. After electrophoresis, the gel was incubated with fixing buffer (50 mM Tris-HCl with pH 8, 20% isopropanol) overnight for removing the SDS. Then the isopropanol was removed by washing the gel with buffer containing 50 mM Tris-HCl (pH 8) and 20% 2-mercaptoethanol twice (25 minutes for each time). The proteins were denatured by incubating with buffer containing guanidine-HCl (50 mM Tris-HCl with pH 8, 6 M guanidine-HCl and 0.3% 2-mercaptoehanol) for 90 minutes. After the incubation, the gel was washed with buffer containing 50 mM Tris-HCl (pH 8), 0.04% Tween 40, 1 mM EDTA and 0.3% 2-mercaptoethanol to remove the guanidine-HCl twice (1 hour for each time). Then the proteins were renatured by incubating with buffer containing 50 mM Tris-HCl (pH 8), 0.04% Tween 40, 1 mM EDTA, 0.3% 2-mercaptoethanol and 3 mM DTT for 1 hour followed by incubating with another fresh aliquot of the same buffer for overnight. All the above procedures were carried out under room temperature. After the serial treatment, the gel was incubated with reaction mixture (50 mM Tris-HCl with pH 8.0, 0.1 mM EGTA, 0.01% Tween 20, 2 mM dithiothreitol, 20 mM MnCl₂, and 1.5 mM DiFMUP) at 37° C. for 10 minutes. Then the gel was put into a transilluminator to detect fluorescent bands with an excitation wavelength at 365 nm.

Myocardium Infarction Area/Area at Risk Determination

At the end of experiment, the animal was sacrificed. We opened the chest cavity and exposed the heart and aorta. The heart was perfused with 0.9% saline from the apex until the coronary vessels and myocardium turning into pale color. After saline perfusion, the LAD knot was tied again. 1 mL of 1% Evans blue dye was delivered into the heart through the aortic root by retrograde injection. The dye uniformly perfused the whole heart except in the area of the heart previously perfused by the occluded coronary artery (area at risk, AAR). Then the heart was quickly removed, washed in ice-cold 0.9% saline followed by frozen at −20° C. for 20 minutes, then cut into slices of 1 mm with heart matrix. The slices were incubated in 1% triphenyl tetrazolium chloride (TTC) solution (pH 7.4) for 15 minutes at 37° C. followed by fixed in 4% paraformaldehyde for 1 hour at room temperature. After fixation, the slices were photographed with a digital camera through a microscopy. The areas stained with Evans blue negative but TTC-positive (AAR), and TTC-negative area (infarct area) were all measured digitally by ImageJ software. We used ratio of infarction area to total AAR to represent the myocardial infarct size.

Serum Troponin I Measurement

Before the mouse was sacrificed, blood was drawn from the facial vein. The blood was put in the room temperature for 30 minutes and then centrifuged at 4° C. for 15 minutes at 5000 rpm. After centrifuging, the serum is collected for troponin I measurement with troponin I ELISA kit (KT-469, Kamiya Biomedical Company) according to the manufacture's protocol.

Neonatal Cardiomyocyte Primary Culture

The heart tissues were taken from 1-3 day-old wild-type C57BL/6 mice neonatal mice. Pierce™ Primary Cardiomyocyte Isolation Kit (Thermo Fisher Scientific) was used in this experiment to isolate the cardiomyocytes. According to the manufacture's instruction, we obtained cardiomyocytes and then maintained them in Dulbecco modified Eagle medium (DMEM) supplemented with 10% fetal bovine serum (FBS), 1% Penicillin/Streptomycin and growth supplement supplied by the kit at 37° C. incubator in a 5% CO₂humid atmosphere.

RNAi and Transfections

Specific Ptpra and Ptpn12 RNA interference were predesigned ON-TARGETplus SMARTpool siRNA purchased from GE Dharmacon™ company. All transfections were performed by Lipofectamine® 2000 (Invitrogen) according to the manufacture's instructions.

Hypoxia/Reoxygenation Protocol and LDH Measurement

Low glucose DMEM without phenol red was used in the H/R experiment to facilitate the following LDH measurement. For the H/R group, the culture medium was pre-incubated in the hypoxic condition (1.5% O₂) created by MiniMACS anaerobic workstation (Don Whitley Scientific) for 24 hours. Then the cells were incubated with the hypoxic DMEM under 1.5% O₂ circumstance for 2 hours. At the end of hypoxic treatment, the culture medium was replaced with fresh DMEM immediately and the cells were moved to the 37° C. humidified normoxic incubator for another 2 hours. In the control group, the cells were maintained in the fresh DMEM in 37° C. humidified normaxic incubator for 4 hours.

For quantifying the H/R injury to the cardiomyocytes, the conditioned medium was collected at the end of hypoxia and reoxygenation and a commercial colorimetric kit (Taraka, #MK401) was utilized to measure the LDH activity in the medium according to the manufacture's instructions.

Immunofluorescence Staining

At the end of treatment, the cells were fixed with 4% paraformadehyde followed by permeabilizing the cell membrane with 0.1% Triton X-100. After blocking with 5% BSA, the cells were incubated with primary antibody specific for troponin T (Thermo Fisher Scientific) overnight at 4° C. After incubating with the primary antibody, the cells were then incubated with secondary antibody for one hour and antibody for DAPI at room temperature.

PTP-PEST Purification

PTP PEST Catalytic Domain construct (Amino Acid:1-300) with 6 His tag at N-terminal were expressed in insect cell Sf9-VECL-01 Cell line, using Bac-to-Bac Baculovirus expression system. Cells were infected with baculovirus at 27° C. and harvested after 65 hours of infection. Post harvesting cell suspension were centrifuged and pellet was stored at −80° C. To purify the protein, pellet was thawed and then dissolved in lysis buffer containing 50 mM Tris-HCl, 500 mM NaCl, 5% Glycerol, pH-7.5, and protease inhibitor cocktail tablet, EDTA free from Roche. Cell were lysed by TS-series cell disrupter (Constant System Ltd.) then lysate was centrifuged at 20000 RPM for 30 minutes at 4° C. using JA25.5 rotor in an Avanti J-26-XPI Centrifuge. Supernatant were collected and incubated with 5 ml of Ni-NTA resin for 2 hours at 4° C. on rotating shaker, following this, resin was washed with 100 ml of wash buffer containing, 50 mM Tris-HCl, 500 mM NaCl, 5% Glycerol, 20 mM Imidazole, pH-7.5 then bound protein from Ni-NTA resin were eluted with elution buffer consisting of 50 mM Tris-HCl, 500 mM NaCl, 5% Glycerol, 350 mM Imidazole, pH-7.5. Eluent from Ni-NTA purification were dialyzed overnight in the presence of 1 mg TEV protease to remove N-terminal His tag against 3 L of dialysis buffer consisting of 50 mM Tris-HCl, 500 mM NaCl, 5% Glycerol, 0.5 mM TCEP, pH-7.5 at 4° C. After dialysis, reverse Ni-NTA purification was performed to separate the protein without tag from protein containing tag. Flowthrough of reverse Ni-NTA, containing protein without tag were applied to size exclusion chromatography (SEC) for further purification. For SEC, HiLoad Superdex 75 16/600 column (GE Healthcare) were used, before applying the protein to column, column was equilibrated with protein storage buffer consisting of 50 mM Tris-HCl, 150 mM NaCl, 10 mM DTT, pH-7.5. After SEC purity of the protein was analyzed by SDS page and quantification was carried out by using NanoDrop Spectrophotometer.

IC 50 Kinetic Assay

To determine IC50 of Auranofin for PTP PEST, phosphatase activity assay was performed using phosphopeptides from Paxillin as a substrate in the presence of Auranofin. Lyophilized powder of phosphopeptides was dissolved in 50 mM Tris-HCl, 150 mM NaCl, pH-7.5 to generate stock solution while Auranofin was dissolved in DMSO for Stock solution. During this assay, protein concentration was fixed to 25 nM and peptide concentration 50 μM, while Auranofin was diluted from 0 μM to 200 μM in 200 μl reaction volume with assay buffer containing 50 mM Tris-HCl, 150 mM NaCl, 10 mM DTT, pH-7.5. The reaction was carried out at 30° C. for 30 minutes then the reaction was stopped by adding 30 μl of phosphate reagent from the phosphate assay kit (BioVision) by following the kit's instruction. The absorbance of the reaction was measured at 650 nm by using Tecan Infinite M1000 Pro instrument. The absorbance value was converted to amount of phosphate produced in reaction based on the standard curve generated from known phosphate concentration. Data generated in the reaction was fitted using nonlinear regression mode and IC50 value were calculated based on the equation function “Log(inhibitor) vs response-variable slope” from Graph Pad Prism 5.0.

Cell Lines

H9C2 cell lines were purchased from ATCC® organization, U.S. Cell cultures were maintained in Dulbecco modified Eagle medium (DMEM) supplemented with 10% fetal bovine serum (FBS) and 1% Penicillin/Streptomycin at 37° C. incubator in a 5% CO₂ humid atmosphere.

Echocardiography

At 1 day and 1 week post the indicated I/R episode, the mice were received echocardiography for evaluating the cardiac function. The mice were anesthetized with avertin by intraperitoneal injection first. Then several parameters, including cardiac morphology, wall motion and function, were examined by an ultrasound machine (Philips, iE33 ultrasound system).

Statistical Analysis

All data were shown as mean±STDEV. The data comparison were determined by unpaired Student t test or ANOVA. The p values less than 0.05 were considered as statistically significant.

Results

Activation of Myocardial PTPs Promotes Cardiac Infarction During I/R

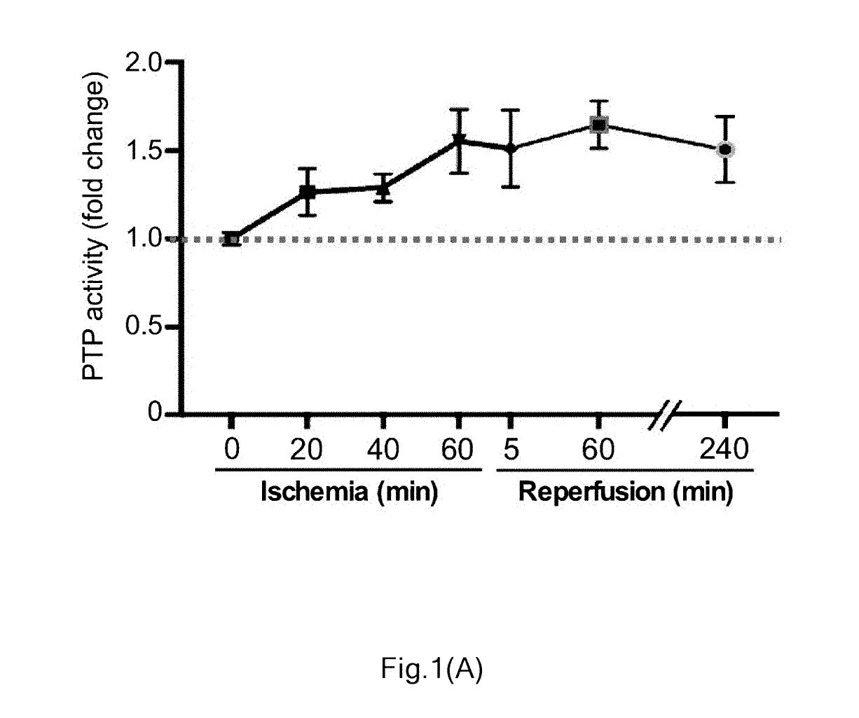

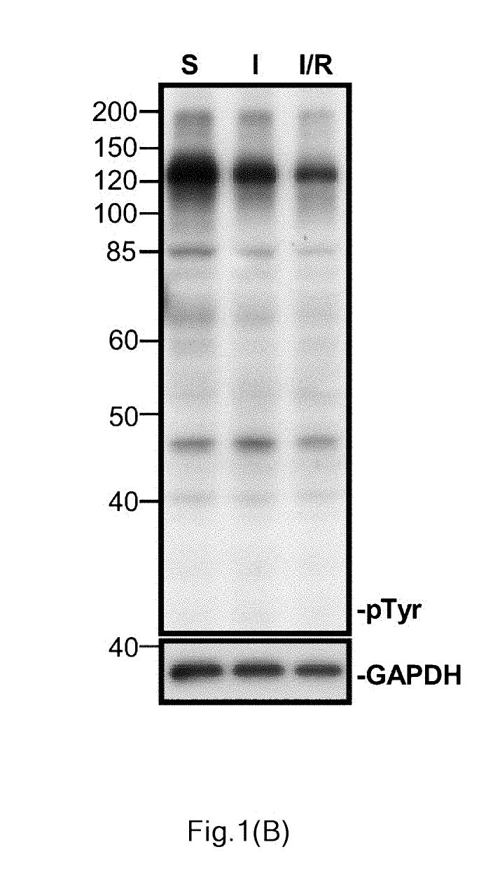

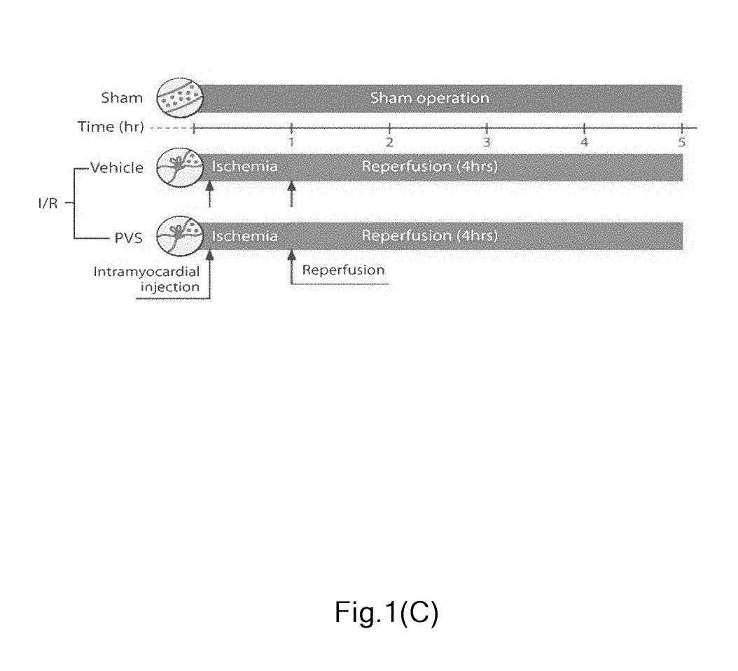

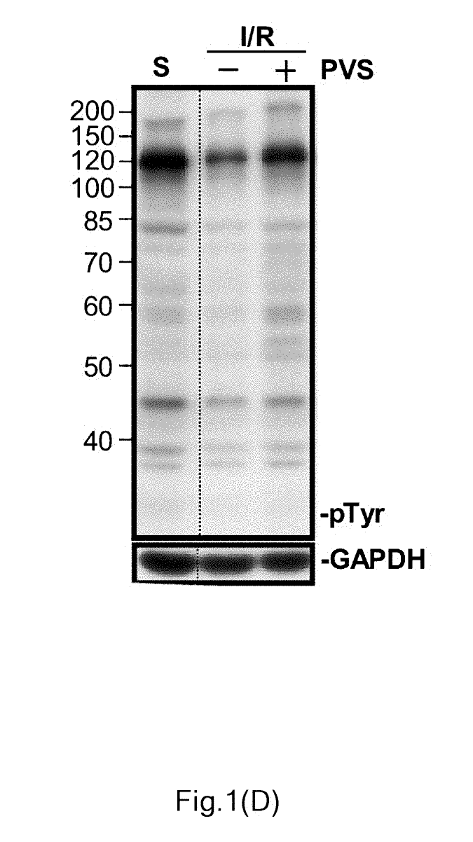

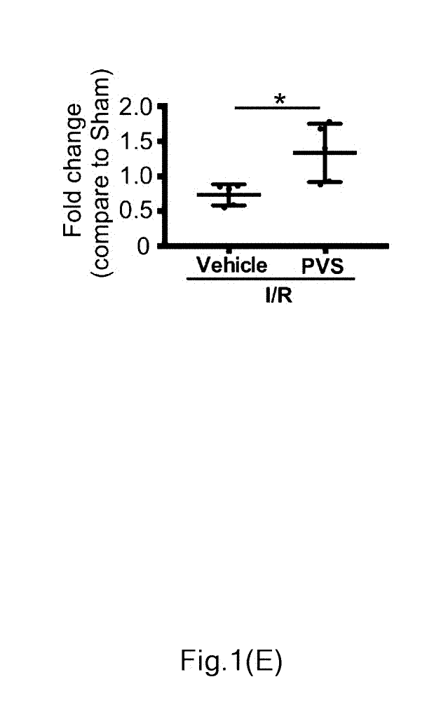

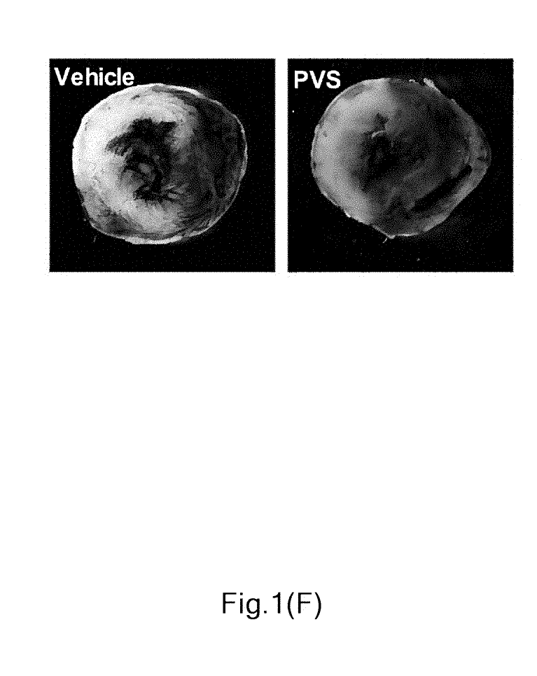

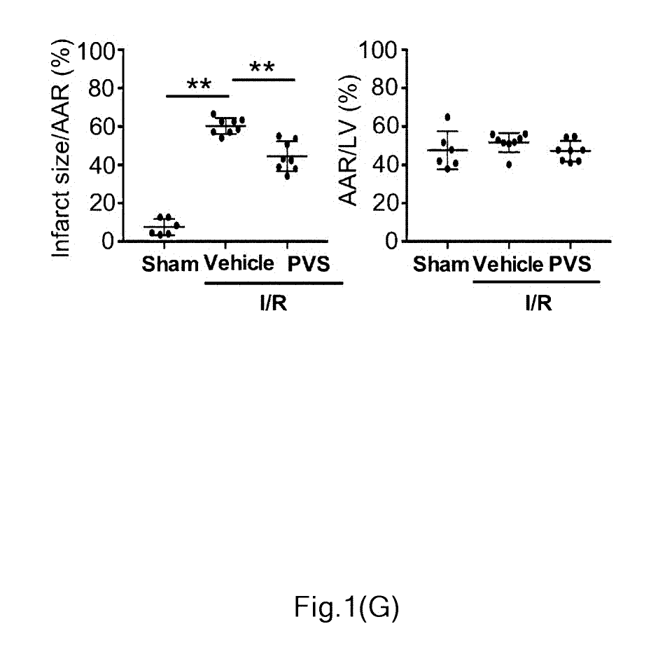

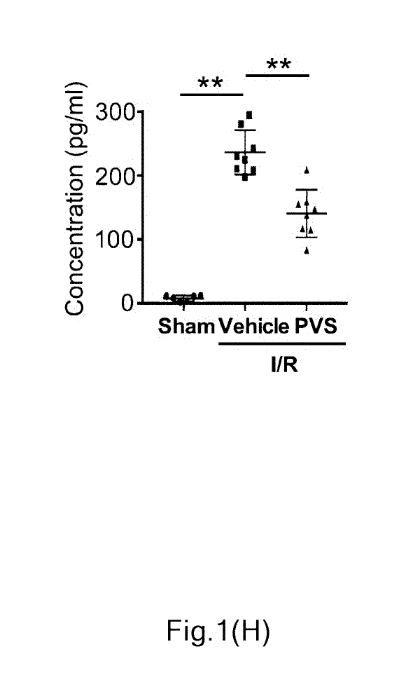

Because protein tyrosine dephosphorylation may be detrimental to the myocardium undergoing I/R, we wanted to study changes in PTP activity during I/R and examined whether such changes would be harmful to the ischemic myocardium. To find out, we measured myocardial PTP activity in three groups of mice: sham, ischemia (I) and I/R. The total extracts were harvested from the ventricles and subjected to the assay that depicts a level of myocardial phosphatase activity as a whole. We focused on the orthovanadate-sensitive fraction of pNPP hydrolysis because this reading represents the activity of tyrosine-specific phosphatases. As shown in FIG. 1(A), overall myocardial PTP activity was significantly elevated in the heart response to ischemia and remained high during the I/R episode. An aliquot of heart extracts was subjected to immunoblotting with anti-pTyr antibody. Myocardial protein tyrosine phosphorylation levels were decreased in both ischemic and I/R episodes (FIG. 1(B)). These results suggested that I- and I/R-induced PTP activation leads to tyrosine dephosphorylation of myocardial proteins.

We proposed that activation of myocardial PTPs might promote cardiac injury during the I/R episode. To test this hypothesis, we used a well-characterized pan-PTP inhibitor, phenyl vinyl sulfone (PVS) to suppress myocardial PTP activity in I/R mouse heart. As illustrated in FIG. 1(C), PVS (5 mg/kg) was administered via intramyocardial injection at the beginning of ischemia, which lasted for one hour followed by four hours of reperfusion. We showed that I/R-caused tyrosine dephosphorylation of myocardial proteins was rebounded by PVS treatment (FIGS. 1(D)-1(E)), in line with the known effect of PVS on PTP inactivation. I/R induced cardiac injury was assessed by evaluating Evans blue/TTC staining of the heart sections and measuring serum troponin I level. Inhibition of PTPs by PVS reduced the area of heart infarction (FIGS. 1(F)-1(G)) and decreased the release of troponin I into the blood (FIG. 1(H)). These results indicated that activation of cardiac PTPs during the ischemia and I/R episodes can harm the myocardium, and that inhibition of PTP activity may protect the heart against I/R injury.

PTP-PEST Aggravates I/R-Induced Injury in Primary Cardiomyocytes

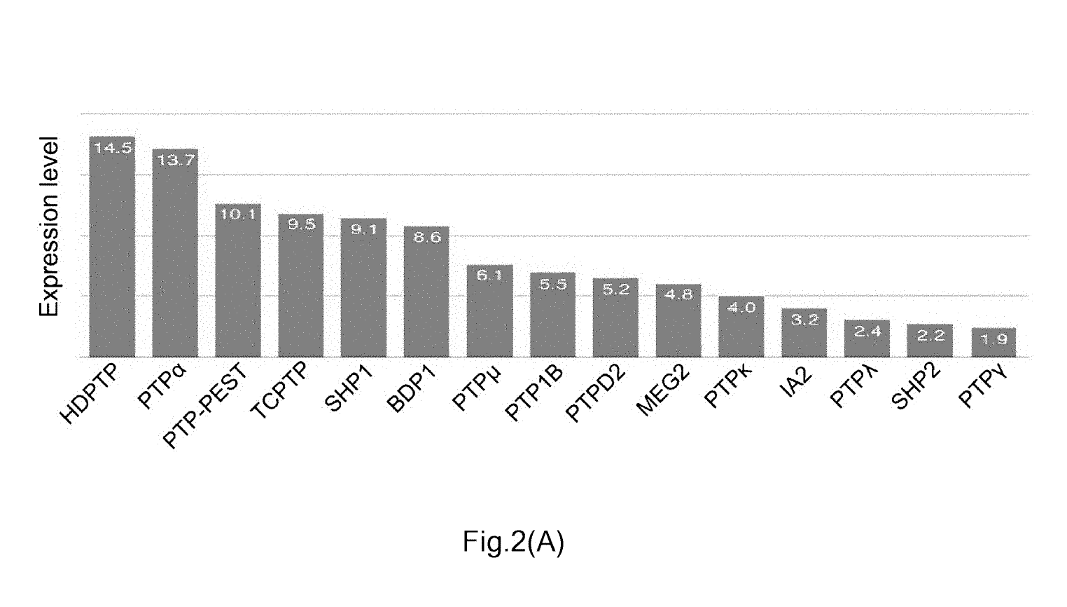

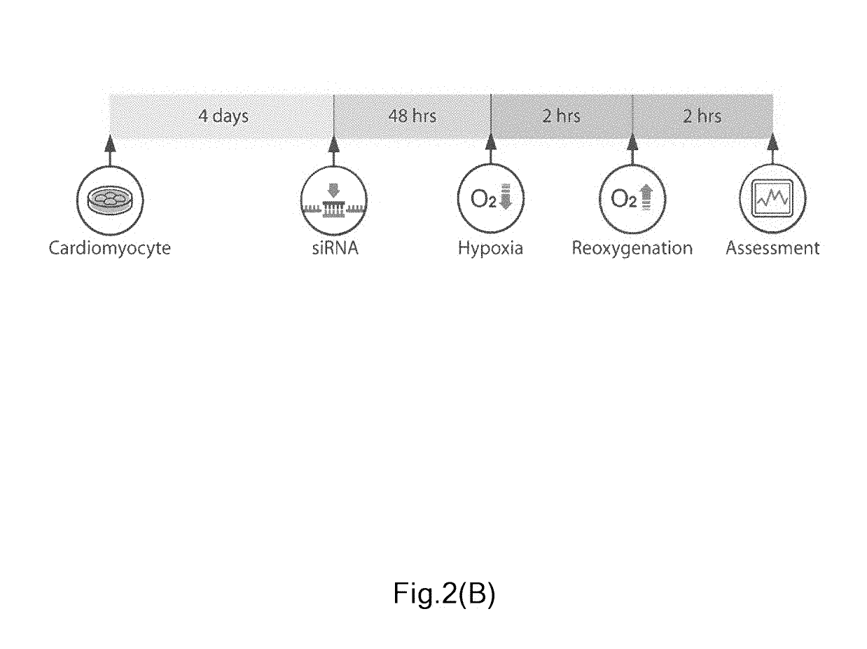

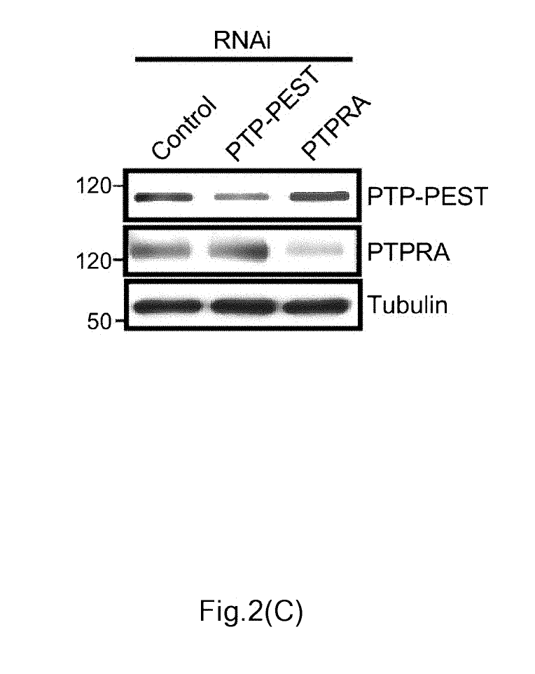

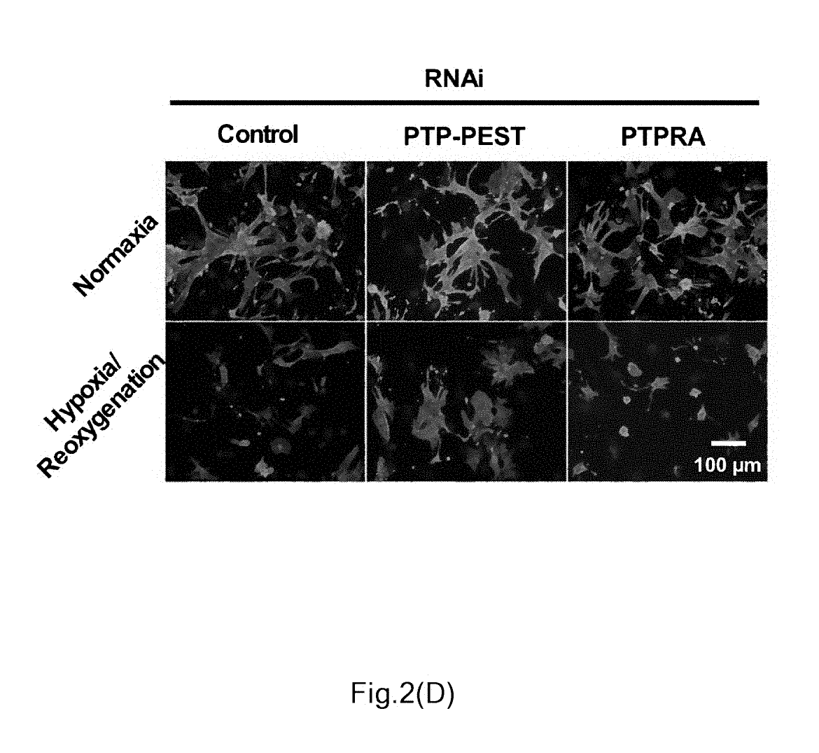

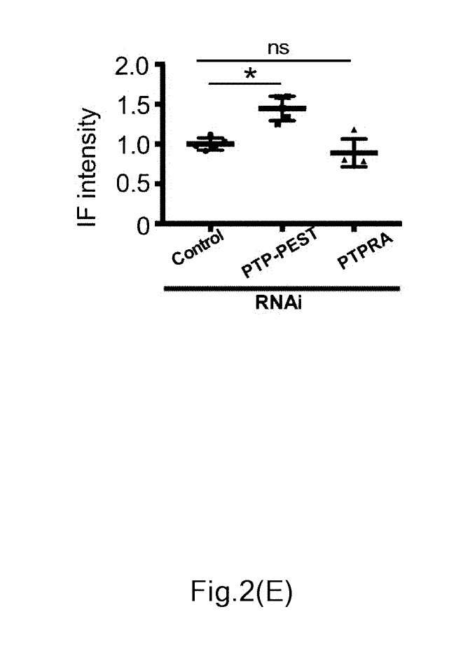

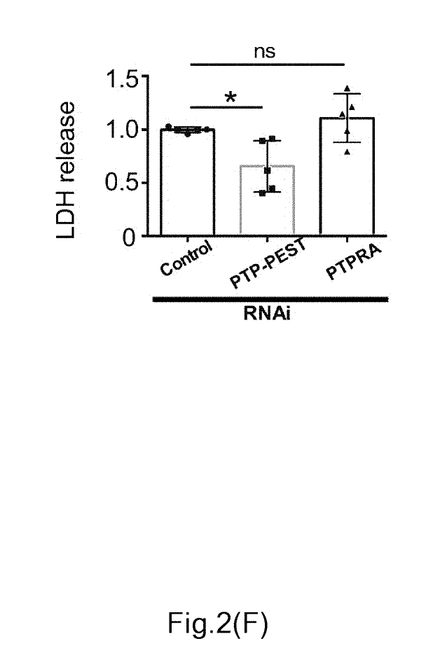

To find PTP candidate involved in the promotion of I/R heart damage, we analyzed RNAseq data (GSM929707) from adult male mouse heart deposited in Gene Expression Omnibus (GEO) datasets. The analysis showed HDPTP, receptor PTP-α (PTPRA), and PTP-PEST to be the three most abundant PTPs in the mouse myocardium (FIG. 2(A)). HDPTP has been previously characterized as an intrinsically inactive phosphatase due to a lack of conserved residues required for catalysis. Thus we ruled out the involvement HDPTP in I/R-induced activation of myocardial phosphatases. We then examined the role of PTP-PEST and PTPRA in cardiac I/R injury. Using neonatal cardiomyocytes as a model, we ablated the expression of endogenous PTP-PEST or PTPRA by specific siRNA oligonucleotides (FIG. 2(B)). The knockdown effect was shown in FIG. 2(C). The population of neonatal cardiomyocytes with low levels of PTP-PEST or PTPRA was then subjected to hypoxic exposure (H) for two hours, followed by reoxygenation (R) for two hours (FIG. 2(B)). At the end of this H/R episode, troponin T staining was performed to that we could observe the morphological change in the cardiomyocytes. The cells in control and PTPRA RNAi groups showed obvious shrinkage in size when exposed to H/R. On the other hand, PTP-PEST knockdown resulted in the resistance of cardiomyocytes to H/R-induced change of morphology (FIGS. 2(D)-2(E)). The release of LDH into the culture medium was also quantitated for monitoring the degree of cell injury. We found that PTP-PEST RNAi group released LDH significantly less than other two groups (FIG. 2(F)). Together, these results suggested that PTP-PEST, likely to be activated during I/R, may exacerbate myocardial injury of the affected heart.

Cardiac PTP-PEST is Cleaved and Subsequently Activated During I/R

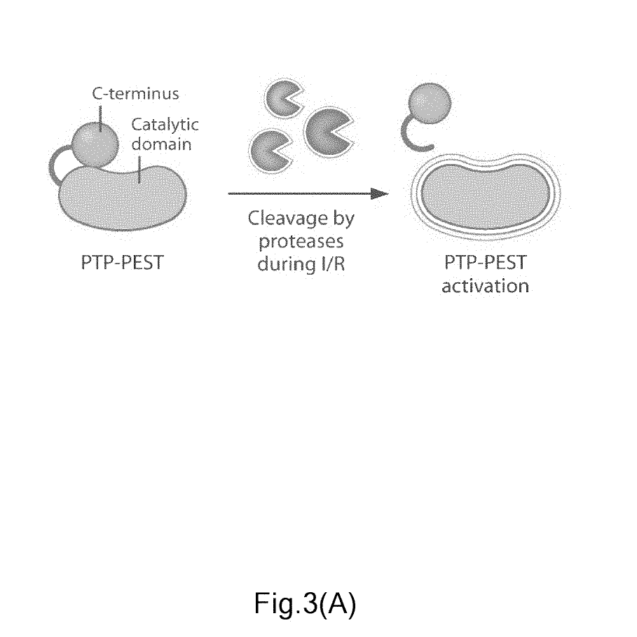

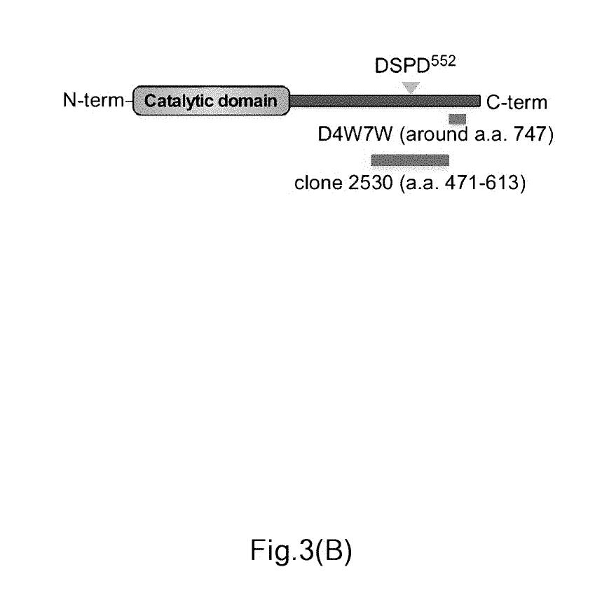

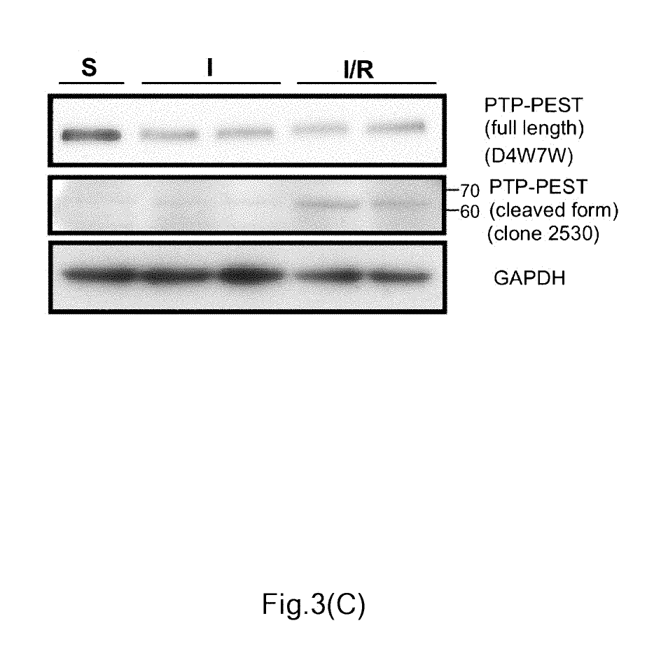

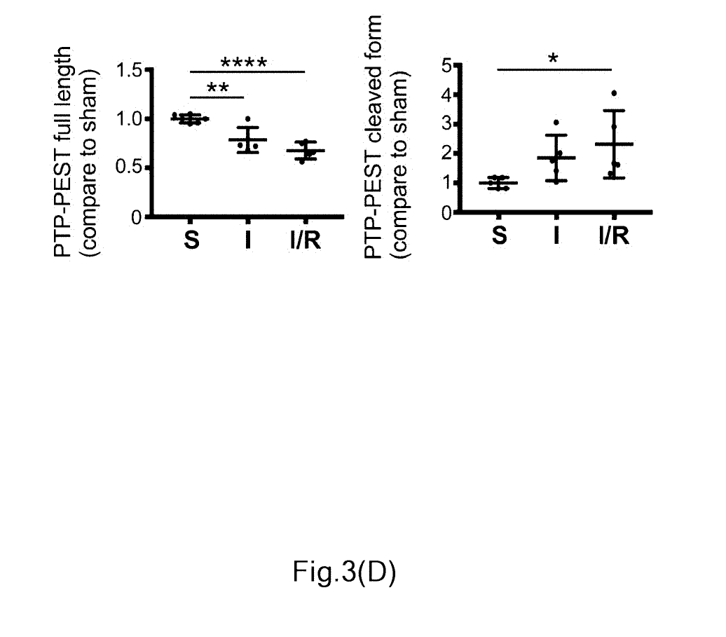

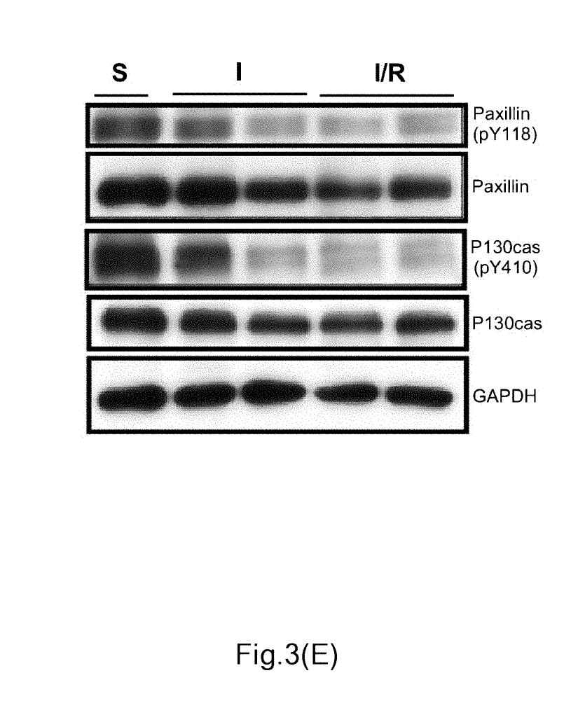

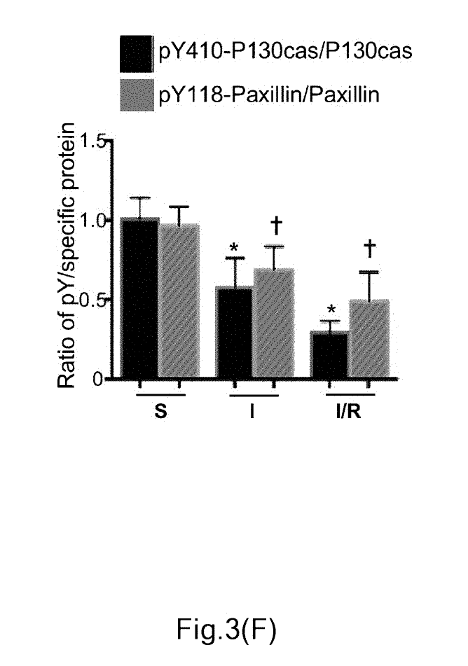

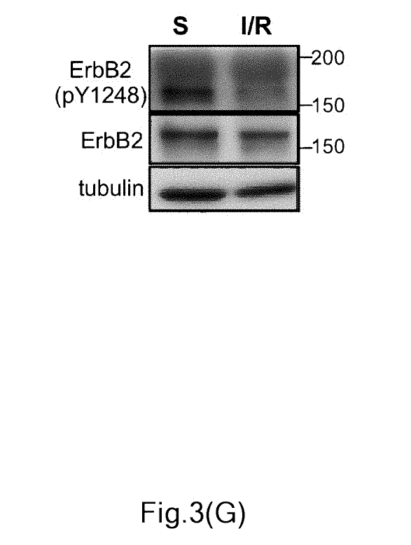

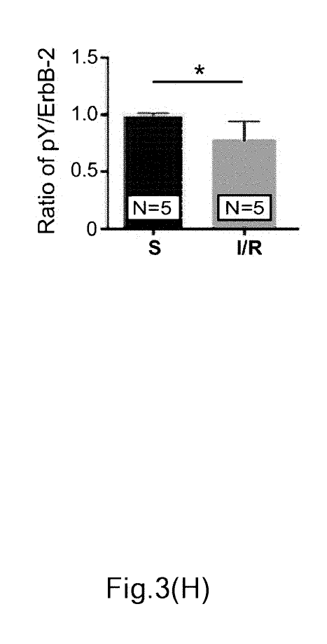

We next explored the mechanism possibly governing the activation of PTP-PEST in the heart under I/R stress. A number of cardiac proteases can be activated during I/R, including caspases and calpain. Interestingly, according to our previous report, PTP-PEST is cleaved by Caspase-3 at the position located in the C-terminal region (D549SPD552) thus releasing the N-terminal phosphatase domain. This truncated form of PTP-PEST exhibits higher enzymatic activity compared to the full-length form. We hypothesized that Caspase-3-mediated removal of Cterminal tail would promote PTP-PEST activity in the heart during I/R (FIG. 3(A)). To test this hypothesis, we probed the total heart extracts from the sham control and the I/R sample with the antibody (clone 2530) that recognizes the cleaved PTP-PEST with an integral phosphatase domain (FIG. 3(B)). Immunoblotting revealed a PTP-PEST variant of ˜62 kDa on SDS-gel in the I/R heart but not in the sham group (FIGS. 3(C) and 3(D)), suggesting the presence of a C-terminally truncated PTP-PEST in response to AMI. Interestingly, the amount of full-length PTP-PEST also showed corresponding decrement in I and I/R (FIGS. 3(C) and 3(D)). Collectively, these findings highlighted that I/R-induced cleavage of PTP-PEST, likely promoted by Caspase-3, lead to the activation of this phosphatase. We then examined whether the well-known substrates of PTP-PEST, including Paxillin, p130Cas and ErbB-2, were tyrosine dephosphorylated during I/R. As shown in FIG. 3(E)-3(H), all three substrates were in a low level of phosphorylation on the specific Tyr residue targeted by PTP-PES in the I/R heart, consistent with the cleavage-induced activation of PTP-PEST.

Auranofin Induces Conformational Changes of PTP-PEST Leading to a Decrease of its Phosphatase Activity

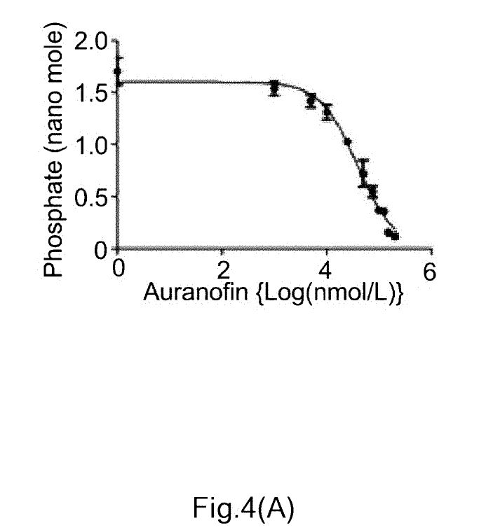

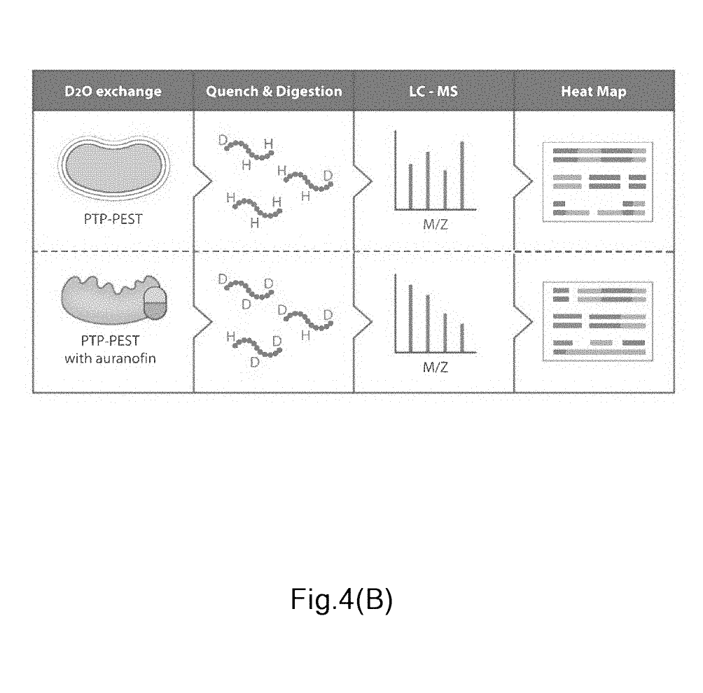

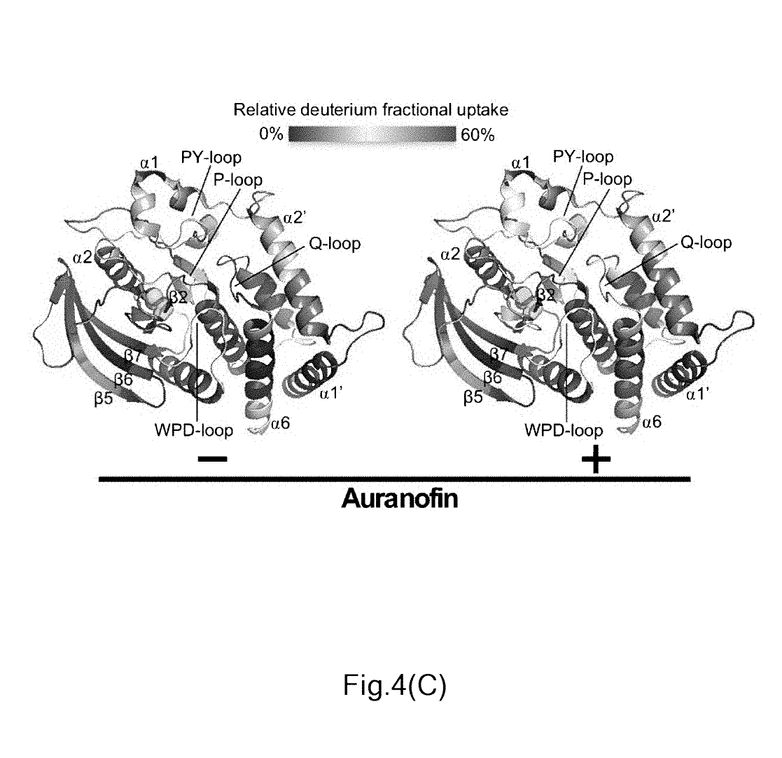

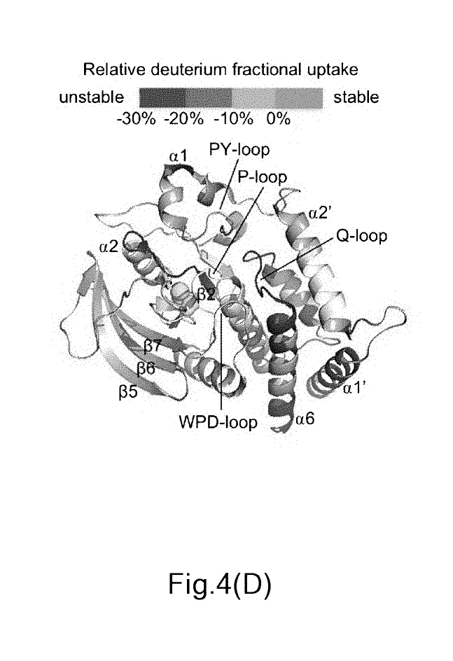

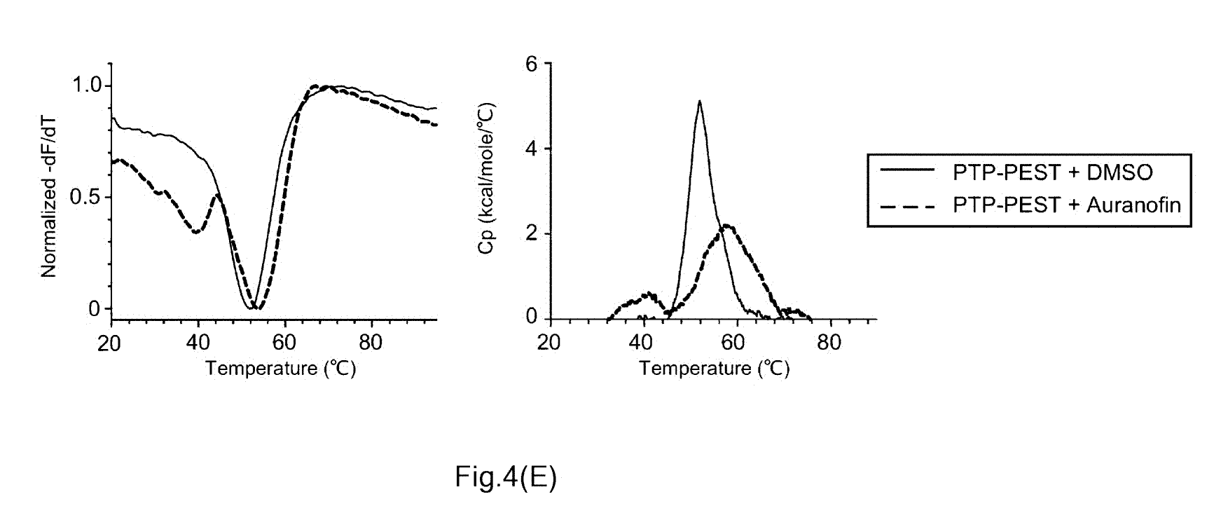

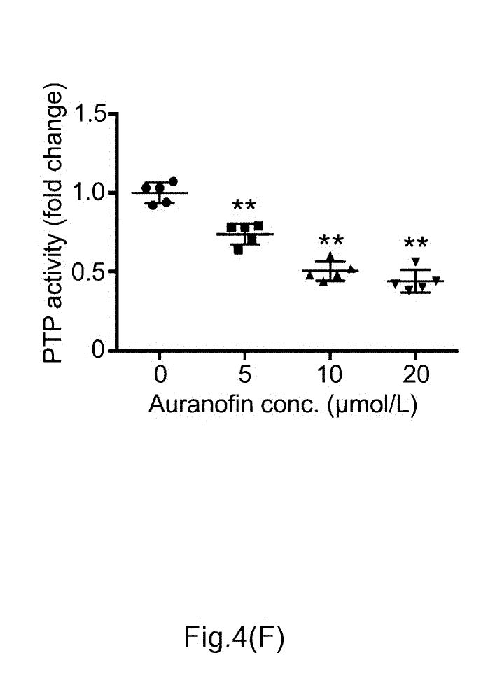

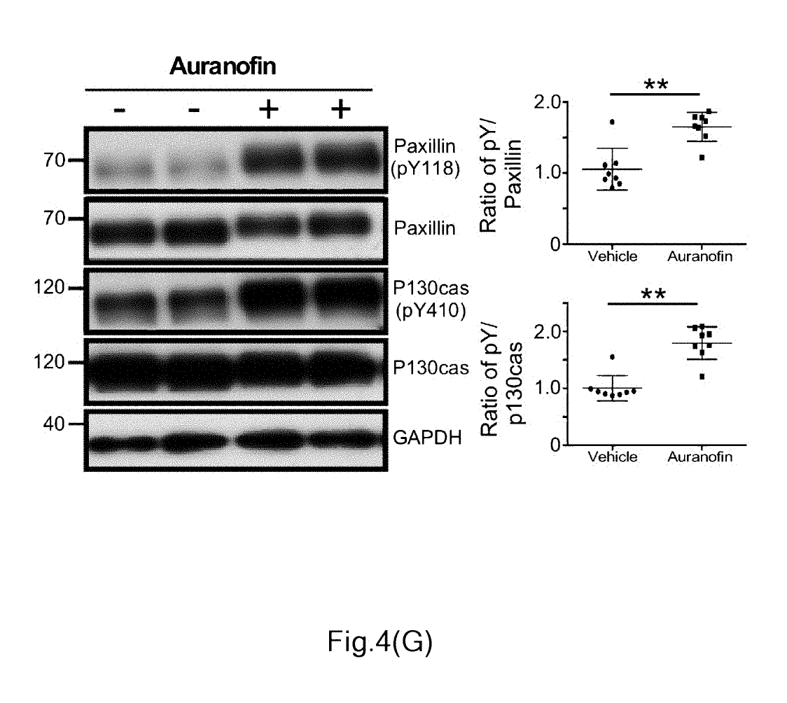

Our results highlighted that activation of PTP-PEST could adversely affect hearts undergoing I/R (FIGS. 2-3). The next step was to explore the possibility of targeting PTP-PEST to attenuate the I/R injury. It was suggested that Auranofin, a drug with potential as a therapy for a number of human diseases, acts to inactivate PTPs and is relatively specific to PTP-PEST. We recapitulated the inhibitory effect of Auranofin on the enzymatic activity of purified PTP-PEST using synthetic phospho-Paxillin peptide as a substrate (IC50=38.7 μmol/L, FIG. 4(A)). To further unravel the molecular basis for Auranofin-induced inactivation of PTP-PEST, we utilized HDX-MS to characterize the internal dynamics of phosphatase domain in the absence or presence of the compound (workflow shown in FIG. 4(B)). We obtained 83 and 84 unique peptides from apo and Auranofin-bound PTP-PEST from the HDX-MS data analyses, corresponding to sequence coverage of 88 or 83%, respectively (detailed MDX-MS report is available in the Supplementary Materials). Structural mapping of the fractional deuterium uptake, a measure of folding stability of individual amino acids, onto the crystal structure of PTP-PEST indicated that Auranofin treatment resulted in increased deuterium uptakes over multiple regions compared to the results of apo PTP-PEST, while reduction in deuterium uptake was limited to the β5-7 (FIG. 4(C)). The impacts of Auranofin treatment on the folding stability of PTP-PEST could be better visualized by examining the difference of fractional deuterium uptake (subtraction between apo and Auranofin-treated; FIG. 4(D)). Detailed examinations of the HDX-MS data revealed that the most significant increase in fractional deuterium uptake occurred in the al helix (M1-K9), the region between β1 and α2 (93F-113T), and the region between α4 and Q-loop (K252-L300), which constitute the key structure surrounding the P loop (FIG. 4(D)). This result indicated that these regions became more flexible after Auranofin treatment. Meanwhile, the three-stranded β-sheet (β5, β6 and β7) exhibited significantly reduced fractional deuterium uptakes (FIG. 4(D)), suggesting a localized stabilizing effect in response to Auranofin treatment. Collectively, the HDX-MS results demonstrated that Auranofin treatment destabilizes a large part of PTP-PEST while it locally stabilizes the β-sheet. To cross-validate the HDX-MS results, we examined the thermal stability of PTP-PEST by differential scanning fluorimetry (FIG. 4(E), left panel) and differential scanning calorimetry (FIG. 4(E), right panel). In both cases, apo PTP-PEST exhibited a single unfolding event with a melting temperature of 51.8° C. and 52.4° C., respectively (black lines in FIG. 4(E)) while two unfolding events were observed in the presence of Auranofin (red lines in FIG. 4(E)). These findings were consistent with the HDX-MS results in that Auranofin disrupted the folding cooperativity of PTP-PEST, leading to destabilization of the region surrounding the P loop while the β-sheet was stabilized (FIG. 4(D)). Collectively, for the first time these results illustrate a potential mechanism through which Auranofin inhibits PTP-PEST via allosteric destabilization effect rather than direct binding to the active-site Cys residue. We propose that increased flexibility of multiple α-helixes near the P loop may affect the substrate binding and access to the phosphatase domain, leading to decreased catalysis of PTP-PEST-mediated tyrosine dephosphorylation. We further treated H9C2 cells with Auranofin at various concentrations (5, 10 and 20 μmol/L) for 20 min. As shown in FIG. 4(F), the overall PTP activity was decreased in H9C2 cells exposed to Auranofin. Consistently, Auranofin induced an increase of phosphorylation on the specific Tyr residue in Paxillin or p130Cas (FIG. 4(G)), likely due to drug-caused inactivation of endogenous PTP-PEST.

Auranofin Protects the Heart from I/R Injury Through Modulation of the PTP-PESTErbB-2 Signaling Pathway

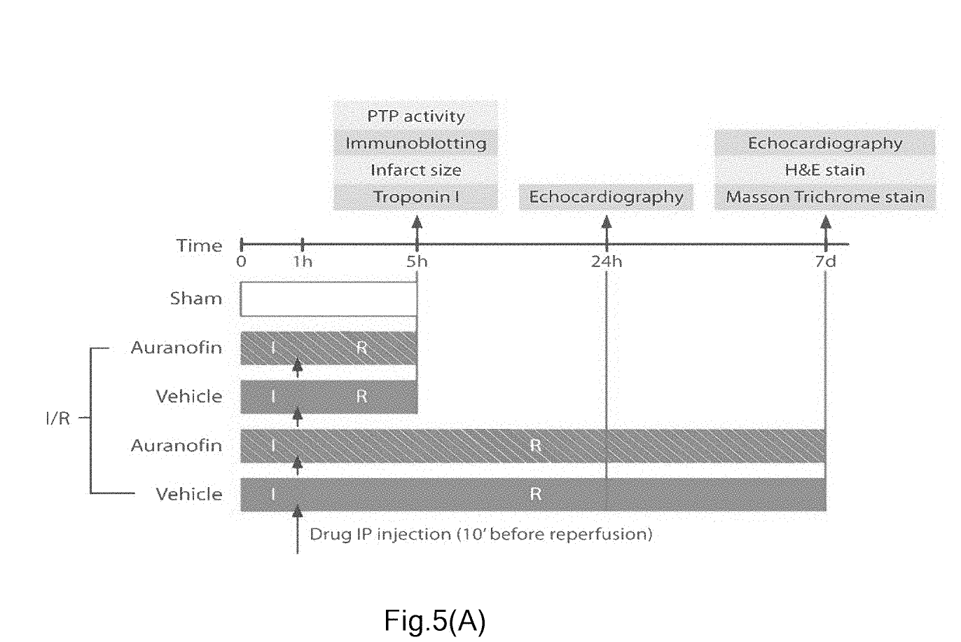

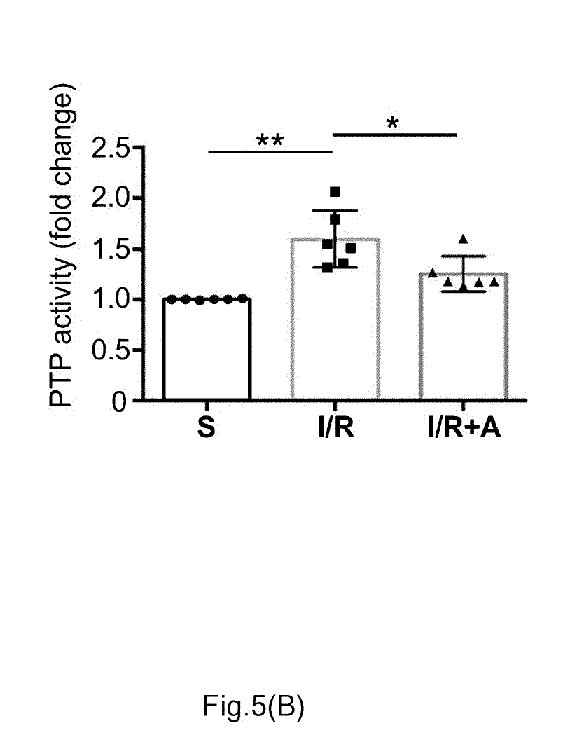

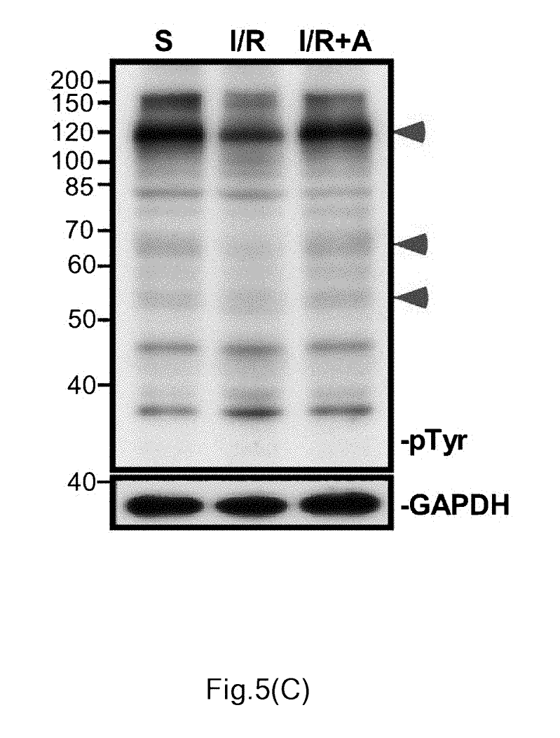

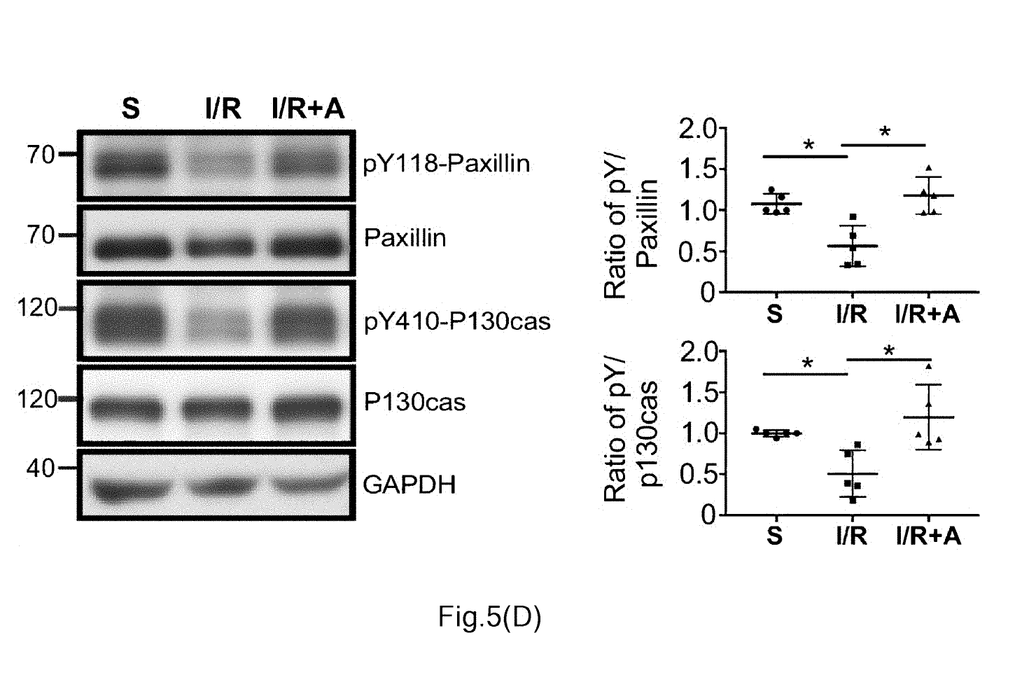

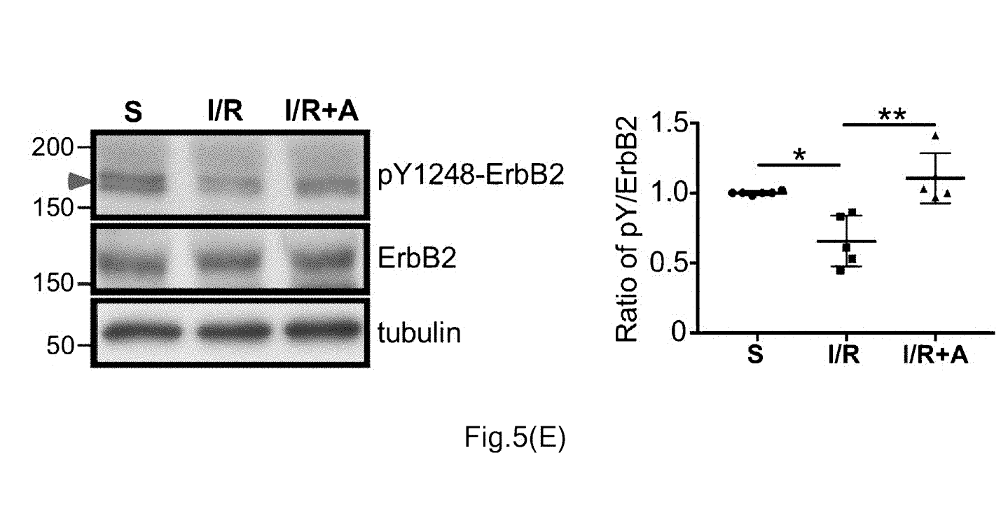

To explore the potential effect of Auranofin on AMI, we applied this drug to I/R mice (experimental design shown in FIG. 5(A)). Intraperitoneal injection (IP) of the animals with Auranofin (2 mg/kg) ten min before reperfusion significantly suppressed overall myocardial PTP activity (FIG. 5(B)). The immunoblotting probed with anti-pTyr antibody also showed that the decreased protein tyrosine phosphorylation in the I/R heart could be reversed by Auranofin (FIG. 5(C)). Specifically, the I/R episode-induced decrease of pTyr signaling in Paxillin and p130Cas was restored by Auranofin (FIG. 5(D)), suggesting that endogenous PTP-PEST might be inactivated in response to the drug treatment. We further examined the phosphorylation level of ErbB-2 at the position of Tyr1248, a known PTP-PEST targeting site that is critical for ErbB-2-mediated survival signaling via downstream Src-dependent pathway, in the heart samples. Notably, Tyr1248 phosphorylation of ErbB-2 was significantly reduced in the I/R heart (FIG. 5(E)). On the other hand, I/R-suppressed phosphorylation of Tyr1248 in ErbB-2 was restored by Auranofin (FIG. 5(E)).

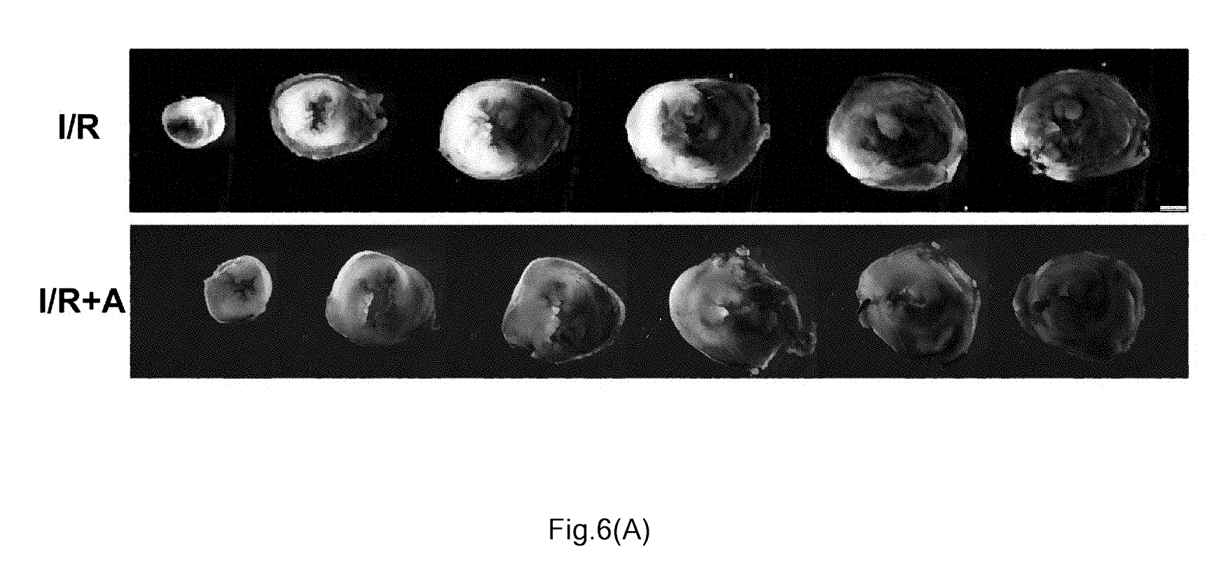

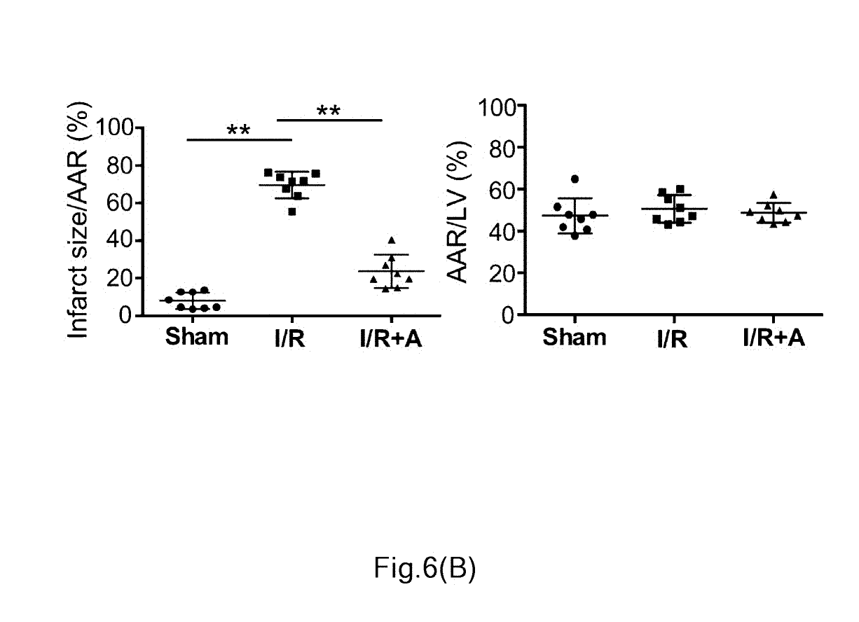

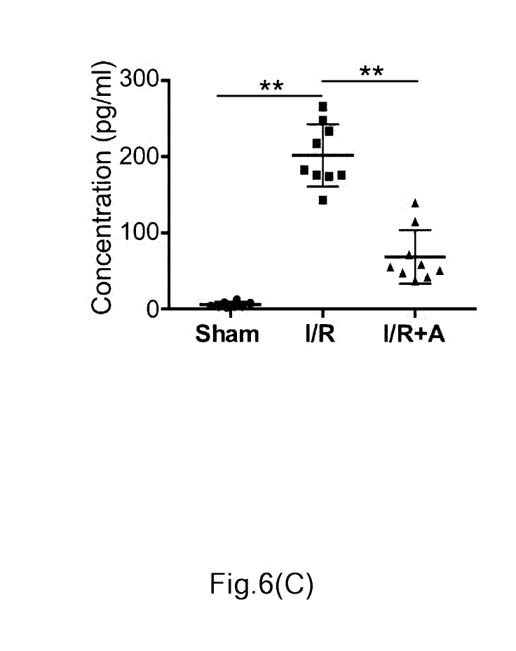

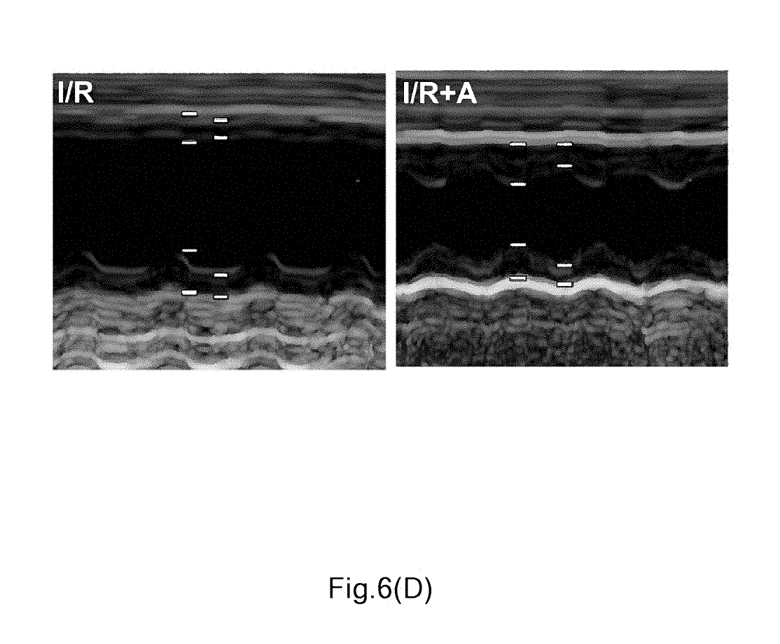

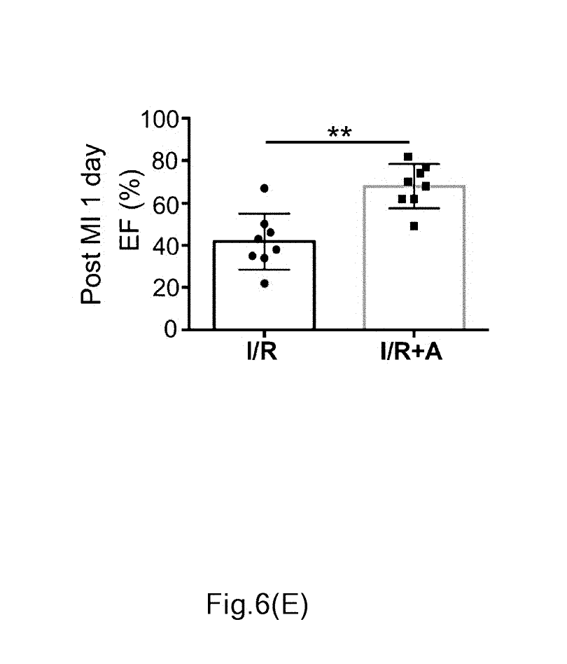

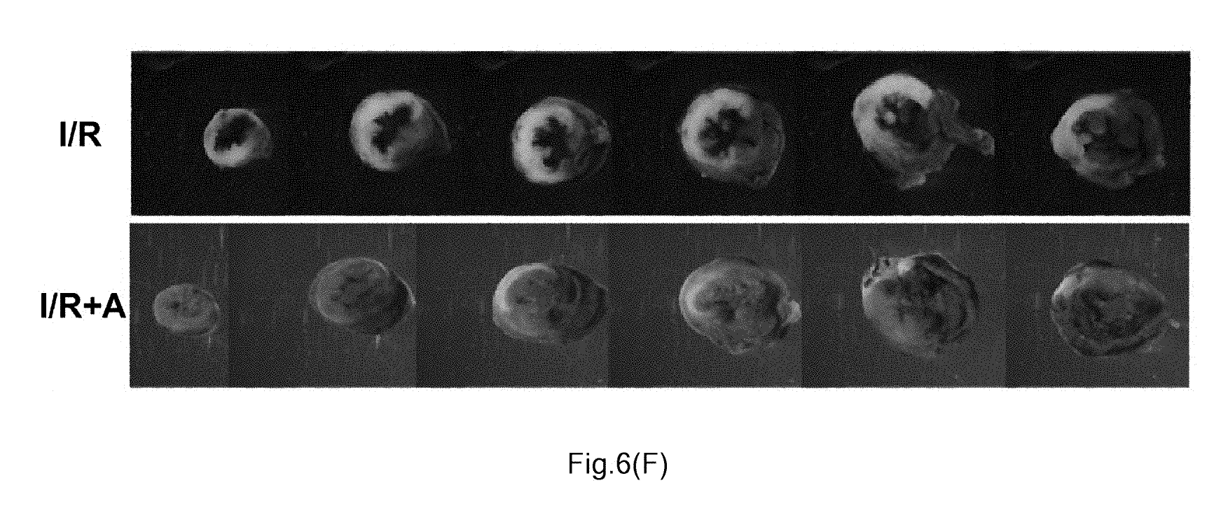

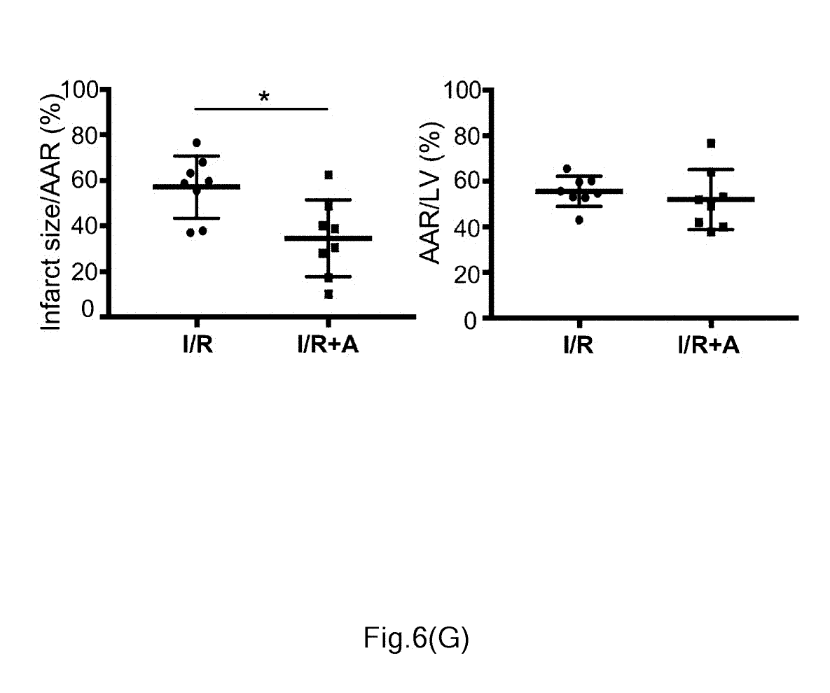

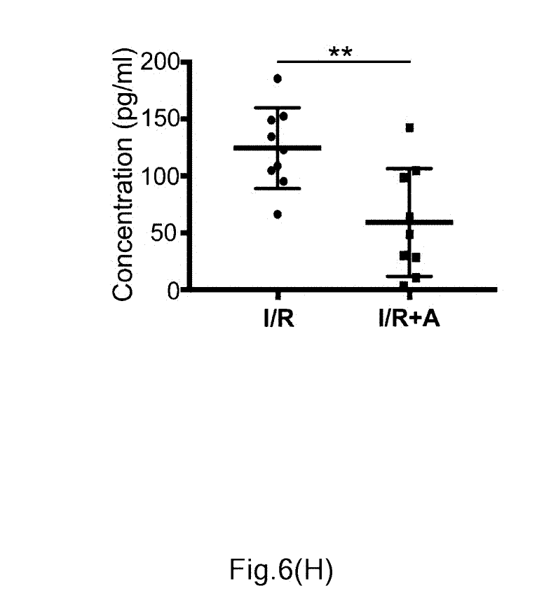

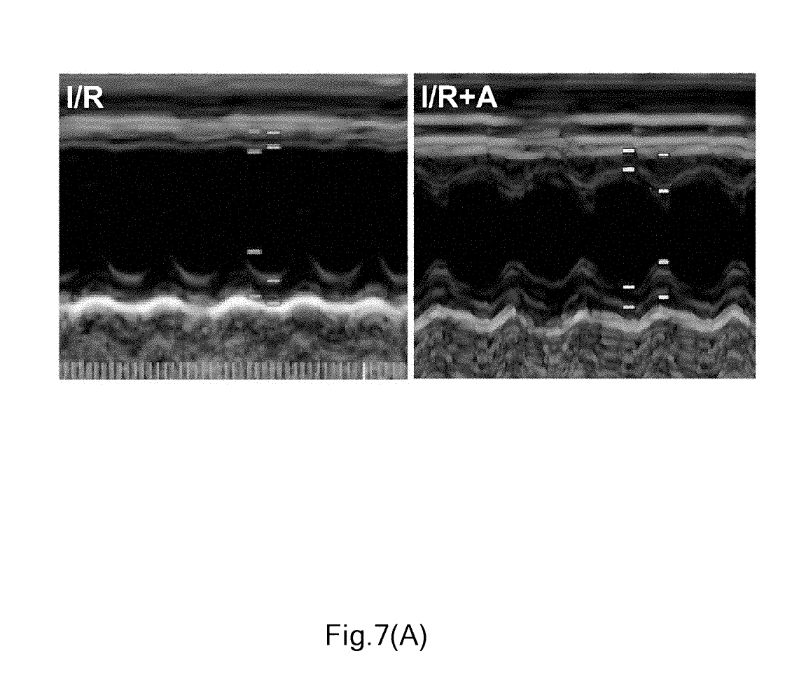

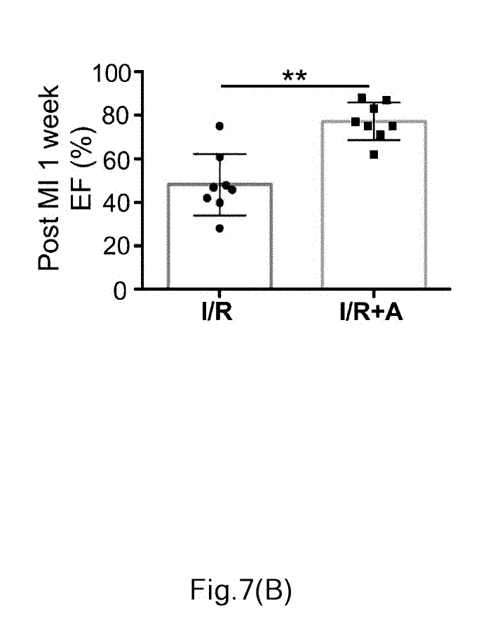

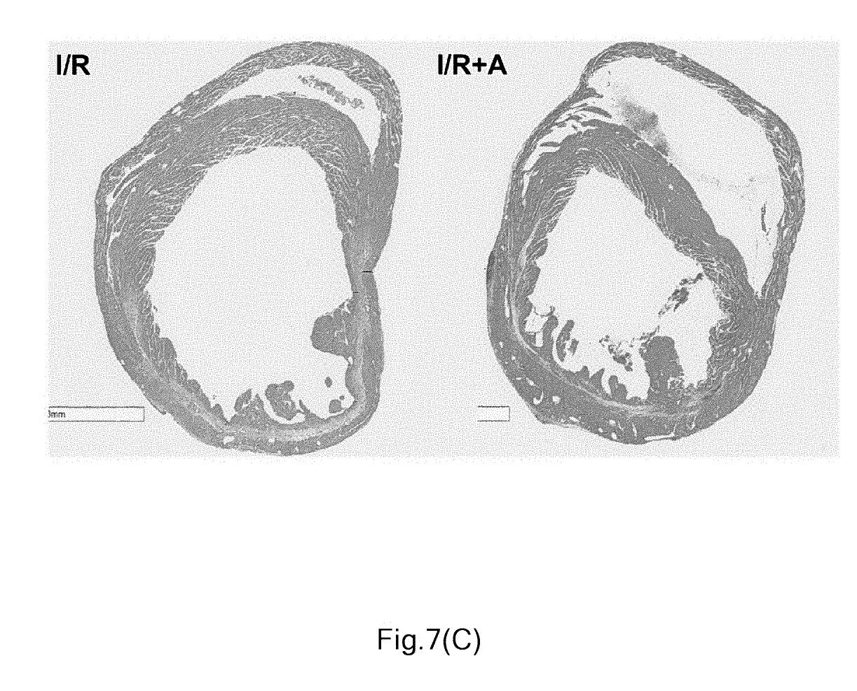

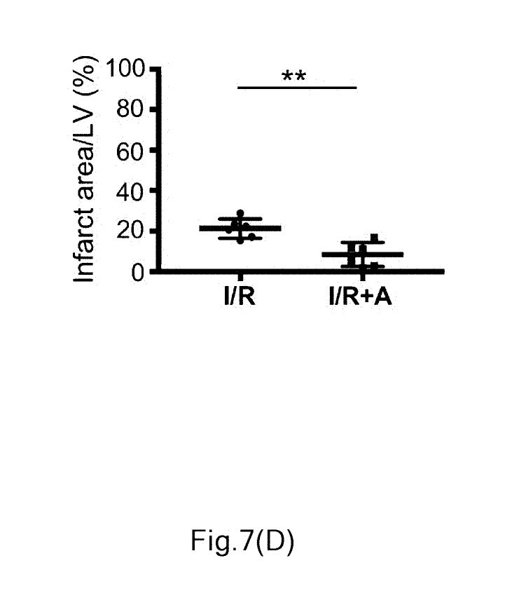

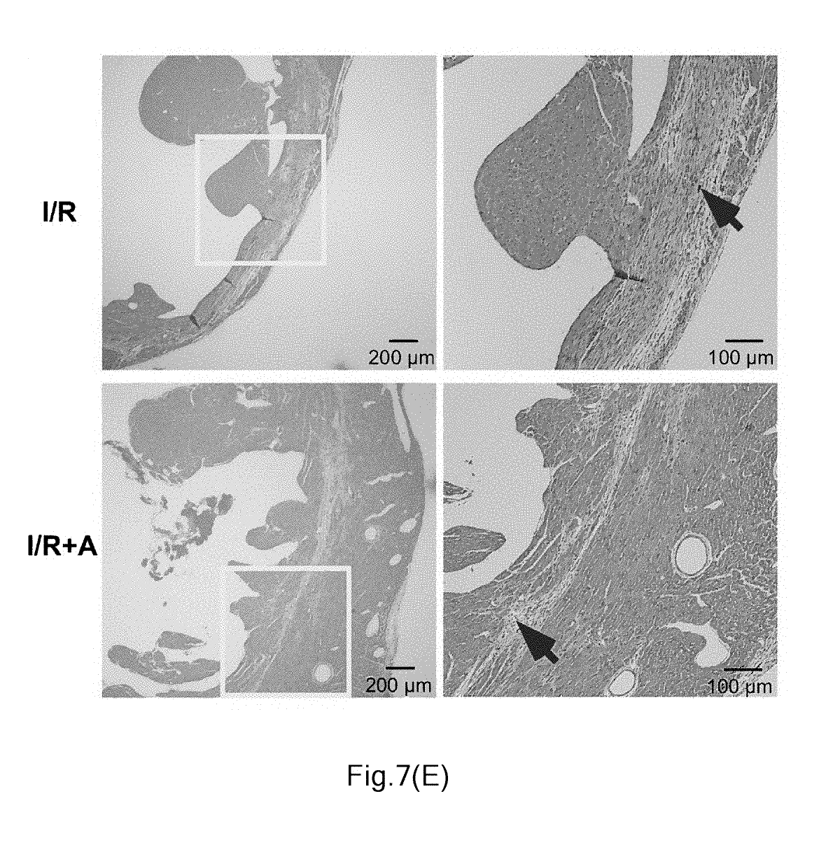

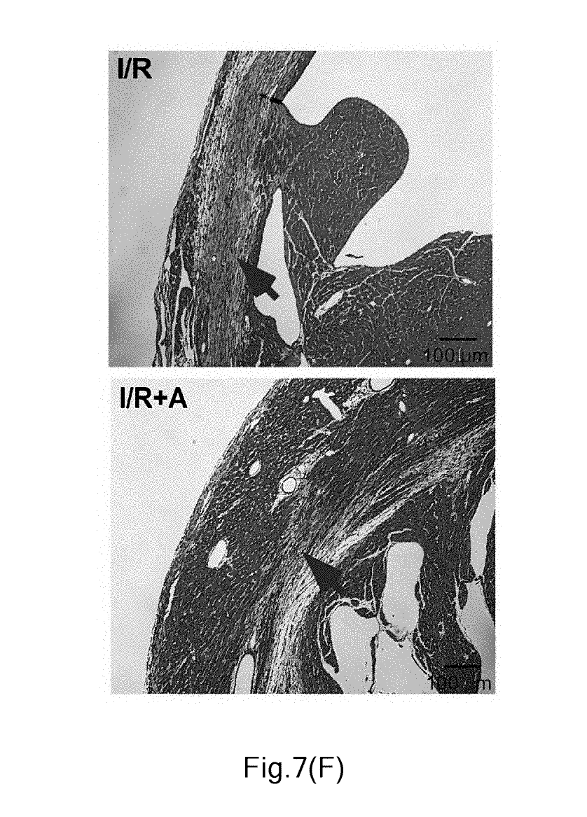

Considering the important role of ErbB-2 in regulating survival of cardiomyocytes, we hypothesized that Auranofin might sustain the cardiac function against the I/R challenge by modulating the PTP-PEST-ErbB-2 signaling axis. To find out, we treated I/R animals with this drug (FIG. 5(A)). Clearly, the infarct size four hours after reperfusion was significantly decreased in the animals that received Auranofin, compared to vehicle-only controls (FIGS. 6(A)-6(B)). Serum troponin I level was also reduced in the Auranofin-treated group (FIG. 6(C)). We next used echocardiography to evaluate cardiac performance of the experimental animals. Cardiac ejection fraction showed significant improvement in the Auranofin-treated group one day post-MI (FIGS. 6(D)-6(E)). The Auranofin-treated animals had smaller infarct size (FIGS. 6(F)-6(G)) and lower serum troponin I level (FIG. 6(H)) one day after reperfusion as well. Remarkably, the beneficial effect of Auranofin on the restoration of cardiac performance judged by echocardiography could be sustained up to one week post-MI (FIGS. 7(A)-7(B)). Consistently, the pathological studies carried out one week after I/R episode showed that the Auranofin-treated mice had smaller infarct size (FIGS. 7(C)-7(D)), fewer infiltrating inflammatory cells, less fibrosis and a minor degree of myocardial thinning over the injured area (FIG. 7(E)-7(F)).

The results of present disclosure demonstrate that the activation of PTPs, especially PTP-PEST, during the process of myocardial I/R. This activation of PTP-PEST is harmful to the ischemic myocardium. When we treated the I/R-mice with auranofin for inhibiting the activity of PTP-PEST, the cardiac infarct size is significantly attenuated. In line with decreasing infarct size, the auranofin-treated I/R-animals also has better cardiac function and lower severity of left ventricular remodeling at one day and one week post the indicated I/R event. The protective effect of auranofin, at least part of it, is from inhibition of PTP-PEST and the subsequent preservation of phosphorylation status of ErbB-2. In this study, our findings provide the first evidence that the consequence of PTP-PEST activation during I/R could be one overlooked factor contributed to myocardial I/R injury. Moreover, we also illustrated the protection effect of auranofin in the I/R process and the mechanisms of how auranofin shields the heart from I/R injury.

In addition, we find that PTP-PEST is cleaved and activated during myocardial I/R. Thus we propose that PTP-PEST is activated during myocardial I/R process through proteases-mediated removal of its C-terminus, which further perturbs downstream pTyr signaling and augments the cardiac injury. Based on the finding in this study, we suggest that ROS burst during reperfusion may not cause significant negative effect on the enzymatic activity of PTP-PEST in the I/R situation. We further suggest that the activation of PTP-PEST would further affect ErbB-2 related signaling pathway and results in detrimental effect on myocardial survival during I/R.

In conclusion, we clarified that the PTP-PEST is crucial in promoting myocardial I/R injury through regulating ErbB-2-related signaling pathway in the present study. Furthermore, we demonstrated the encouraging therapeutic effects of auranofin in the disease context of acute myocardial I/R injury for the first time. These results may provide valuable information for managing the patients with myocardial reperfusion injury and improving patients' outcome in the near future.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

### REFERENCES

The references listed below and referred to herein are hereby incorporated into this specification by reference unless this specification expressly provides otherwise.
1. Yellon, D. M. and D. J. Hausenloy, Myocardial reperfusion injury. N Engl J Med, 2007. 357(11): p. 1121-35.
2. Jennings, R. B., et al., Myocardial necrosis induced by temporary occlusion of a coronary artery in the dog. Arch Pathol, 1960. 70: p. 68-78.

1. A method for attenuating or reducing myocardial reperfusion injury in a subject with ischemia myocardium, comprising:
administering to the subject an effective amount of a protein tyrosine phosphatases (PTPs) inhibitor or a protein tyrosine kinases (PTKs) activator.

2. The method of claim 1, wherein a myocardial PTPs activity of the subject is elevated during ischemia and reperfusion.

3. The method of claim 2, wherein a myocardial protein tyrosine phosphorylation of the subject is decreased.

4. (canceled)

5. The method of claim 1, wherein a phosphorylation on Paxillin, p130cas, and ErbB-2 of the subject is restored after the administration.

6. The method of claim 5, wherein the phosphorylation is at Y118 of Paxillin, Y410 of p130cas, and Y1248 of ErbB-2.

7. The method of claim 1, wherein the PTPs inhibitor comprises auranofin, phenyl vinyl sulfone (PVS), or orthovanadate.

8. (canceled)

9. The method of claim 1, wherein the myocardial reperfusion injury comprises cell swelling, contracture of myofibrils or disruption of sarcolemma in a myocardium under ischemic stress.

10. The method of claim 1, wherein the PTPs inhibitor is administered before ischemia, during ischemia or before reperfusion.

11. (canceled)

12. (canceled)

13. The method of claim 1, wherein the subject is a human or an animal.

14. The method of claim 1, wherein the administering to the subject is by at least one mode selected from the group consisting of parenteral, subcutaneous, intra-muscular, intra-venous, intra-articular, intra-bronchial, intra-abdominal, intra-capsular, intra-cartilaginous, intra-cavitary, intra-celial, intra-cerebellar, intra-cerebroventricular, intra-colic, intra-cervical, intra-gastric, intra-hepatic, intra-myocardial, intra-osteal, intra-pelvic, intra-pericardiac, intra-peritoneal, intra-pleural, intra-prostatic, intra-pulmonary, intra-rectal, intra-renal, intra-retinal, intra-spinal, intra-synovial, intra-thoracic, intra-uterine, intra-vesical, bolus, vaginal, rectal, buccal, sublingual, intra-nasal, transdermal, and intra-coronary.

15. The method of claim 1, wherein the effective amount is 0.01-80, 0.05-40 or 0.1-10 mg/kg.

16. (canceled)

17. (canceled)

18. The method of claim 1, wherein a serum troponin I level and an infarct size of the subject are reduced after the administration, and/or an inflammatory cells infiltration, fibrosis and myocardial thinning of the subject are reduced after the administration.

19. (canceled)

20. The method of claim 1, wherein the PTPs inhibitor or the PTKs activator is a composition with a pharmaceutically acceptable carrier comprising solvent, emulsifier, suspending agent, decomposer, binding agent, excipient, stabilizing agent, chelating agent, diluents, gelling agent, preservative, lubricant, or a combination thereof.

21. The method of claim 20, wherein the pharmaceutically acceptable carrier is selected to provide the composition in a form selected from the group consisting of a solution, a suspension, a gel and an ointment.

22. A method for myocardial reperfusion, comprising
administering to a subject in need an effective amount of a protein tyrosine phosphatases (PTPs) inhibitor or a protein tyrosine kinases (PTKs) activator, and
performing a reperfusion therapy on the subject.

23. The method of claim 22, wherein the reperfusion therapy is administered to the subject in need thrombolytic agents or fibrinolytic agents for thrombolysis, and the thrombolytic agents or the fibrinolytic agents comprise streptokinase, urokinase, alteplase, reteplase, tenecteplase, or recombinant tissue plasminogen activator (rtPA).

24. (canceled)

25. The method of claim 23, wherein the reperfusion therapy further comprising administering to the subject in need an anticoagulant including heparin or low molecular weight heparin.

26. The method of claim 22, wherein the reperfusion therapy is a percutaneous coronary intervention (PCI) and a coronary angioplasty, and/or is bypass surgeries that graft arteries around blockages.

27. (canceled)

28. The method of claim 22, wherein the PTPs inhibitor comprises PTP-PEST inhibitor comprising auranofin, or phenyl vinyl sulfone (PVS), or orthovanadate.

29. (canceled)

30. A kit for myocardial reperfusion, comprising:
a protein tyrosine phosphatases (PTPs) inhibitor or a protein tyrosine kinases (PTKs) activator, and
a means for myocardial reperfusion.

31. The kit of claim 30, wherein the PTPs inhibitor comprises PTP-PEST inhibitor comprising auranofin, or phenyl vinyl sulfone (PVS), or orthovanadate.

32. (canceled)

33. The kit of claim 30, wherein the means for myocardial reperfusion is thrombolytic agents or fibrinolytic agents for thrombolysis, and the thrombolytic agents or the fibrinolytic agents comprise streptokinase, urokinase, alteplase, reteplase, tenecteplase, or recombinant tissue plasminogen activator (rtPA).

34. (canceled)

35. The kit of claim 33, further comprising an anticoagulant including heparin or low molecular weight heparin.

36. The kit of claim 30, wherein the means for myocardial reperfusion is selected from the group consisting of a stent, a balloon, aspiration thrombectomy, rotational atherectomy, laser angioplasty, cutting balloon angioplasty, and embolic protection device and any combinations thereof, for a percutaneous coronary intervention (PCI) and a coronary angioplasty, and/or is surgical equipments for bypass surgeries that graft arteries around blockages.

37. (canceled)
